(19) United States
(12) Patent Application Publication
(10) Pub. No.: US 2005/0267396 A1
(43) Pub. Date: Dec. 1, 2005

(54) System and method for the rotatable selection, preparation and recollection of hemodialysis cannulation sites

(76) Inventor: Michael Dame, Cohoes, NY (US)

Correspondence Address: SANDER RABIN MD JD;CONVERGENT TECHNOLOGY PATENT LAW GROUP, 2 IRVING PLACE, TROY, NY, 12180-4417, US

(21) Appl. No.: 10/846,191

(22) Filed: May 14, 2004

(51) Int. Cl. A61M 37/00

(52) U.S. Cl. 604/4.01; 604/116; 128/898

(57) ABSTRACT

The invention is a device and corresponding method for the rotatable selection, preparation and recollection of hemodialysis cannulation sites along a surgically constructed arterio-venous fistula or arterio-venous graft of a patient with end-stage renal disease. The device comprises a program, a site preparation card, transparent dressings held in a dressing folder; transferably detachable self-adhesive mnemonic markers, and a skin marking device. The program has means for removably receiving the site preparation card, the transparent dressings held in the dressing folder, and the detachable self-adhesive mnemonic marker. The skin marking device has means for removably attaching to the program. The method utilizes a system of instruction and interaction with the device to enable the patient to rotatably select, prepare and recall hemodialysis cannulation sites along the patient's arterio-venous fistula or arterio-venous graft, thereby minimizing trauma, infection and pain, and also maximizing the longevity of the arterio-venous fistula or arterio-venous graft.

### BACKGROUND OF THE INVENTION

1. Technical Field

The present invention relates generally to the treatment of end-stage renal disease and, in particular, to the minimization of trauma, infection and pain in patients undergoing hemodialysis and the extension of the longevity of surgical arterio-venous grafts and fistulas used in hemodialysis.

2. Related Art

Hemodialysis is a recurrent chronic treatment indicated for patients with end-stage renal disease (“ESRD”), in which blood is circulated outside of the body of an ESRD patient and through a hemodialyzer for the removal of urea and other molecular and electrolytic blood-borne waste products. In patients with ESRD, hemodialysis is performed about three times a week, with each treatment lasting 3 to 4 hours.

Blood flow rates of about 300 to 400 L/min are necessary for hemodialysis. Venous blood flow is insufficiently slow, and repeated puncture of a large artery is not medically feasible. Consequently, prior to undergoing hemodialysis, a patient with ESRD must have a high-flow arterio-venous (“AV”) access site (hereinafter “hemodialysis access configuration”) surgically constructed, along which, at hemodialysis cannulation sites, paired arterial and venous needles are inserted for withdrawing toxin-laden blood and returning freshly-dialyzed blood.

The preferred method for securing high-flow access to a patient's circulation for hemodialysis is through a surgically created AV fistula (“AVF”), which is often made at the wrist between the radial artery and cephalic vein, or at the elbow between the brachial artery and the basilic vein. If a native vein is unavailable because of fibrosis or atrophy due to prior needling, phlebitis or other injury, a prosthetic vascular graft bridging an artery and a nearby vein (hereinafter an “AVG”) may be utilized. The AVG is generally made of polytetrafluoroethylene. The AVG is commonly either a straight AVG (“SAVG”) or a looped AVG (“LAVG)

Unfortunately, thrombosis, infection and aneurysm formation commonly occur in AVGs and comprise the leading cause of hospital admission in patients with ESRD. There is also a high incidence of septicemia and septic embolization associated with AVG infections. Despite these disadvantages, AVGs are currently the most common way of achieving access to the circulation of an ESRD patient for hemodialysis.

A dialysis treatment occurs over 3 to 4 hours and is generally repeated 3 times a week, typically on either a Monday-Wednesday-Friday schedule or a Tuesday-Thursday-Saturday schedule. At the commencement of each hemodialysis treatment, an efferent needle is inserted into the arterial aspect of the AVG, and an afferent needle is inserted into the venous aspect of the AVG. The efferent needle is connected to tubing that draws blood into the hemodialyzer from the ESRD patient and the afferent needle is connected to tubing that returns blood from the hemodialyzer to the ESRD patient.

After each hemodialysis treatment the needles are withdrawn and a pressure bandage is placed on access/cannulation site. Thereafter, additional gauze is applied to the wound under pressure until bleeding stops. Cessation of bleeding takes about 10 to 20 minutes, at which point the blood-soaked gauze is removed and the wound site is dressed with fresh gauze that is secured by a sure seal band-aid. The patient may then return home. The gauze dressing may be removed several hours after its application, but the sure seal band-aid may be left in place for as long as 8 hours.

The current procedures for preparing an ESRD patient for a hemodialysis treatment omit steps to avoid piercing the same area of a patient's skin before the wounds made by prior piercing to obtain AV access have healed. This omission results in increased cutaneous, subcutaneous and vascular trauma and increased pain.

Chronic repeated insertion of these needles eventuates in traumatic breakdown of the skin, and traumatic breakdown and stenosis of the graft site, particularly in the vicinity of its venous anastomosis. Traumatic breakdown and stenosis requires thrombectomies, AVG salvage, surgical revision procedures and new surgical constructions. AVG life may be prolonged with patch angioplasty at venous outflow stenoses or by adding a new segment of PTFE to bypass areas of venous stenosis.

ESRD patients are also at risk for infection, which is second only to cardiovascular disease as a cause of death in these patients. AVGs have been identified as a major risk factor for septicemia and complications such as endocarditis and septic arthritis in patients undergoing hemodialysis.

Accordingly there is a need for systems and methods that: minimize cutaneous, subcutaneous and vascular trauma; minimize the risk of cutaneous, subcutaneous, vascular and systemic infection; minimize pain; and maximize the functional integrity and longevity of AVGs.

### SUMMARY OF THE INVENTION

The invention is a device and method for the rotatable selection, preparation and recollection of hemodialysis cannulation sites along a hemodialysis access configuration of a patient with ESRD, comprising: a program having an inner face and an outer face; a site preparation card; at least two transparent dressing held in a dressing folder; at least two transferably detachable self-adhesive mnemonic markers; and, a skin marking device having means for its removable attachment to the program, wherein the inner face of the program has means for the removable receipt of the site preparation card and for the removable receipt of the transparent dressings held in the dressing folder, and means for the removable receipt of the transferably detachable self-adhesive mnemonic markers.

### BRIEF DESCRIPTION OF THE DRAWINGS

FIG. 1 is a schematic illustration of a hemodialysis access configuration in the form of an arterio-venous fistula.

FIG. 2 is a schematic illustration of a hemodialysis access configuration in the form of an straight arterio-venous graft.

FIG. 3 is a schematic illustration of a hemodialysis access configuration in the form of an looped arterio-venous graft.

FIG. 4 is a schematic perspective illustration of the invention.

FIG. 5 is a schematic illustration of a skin marking device.

FIG. 6 is a schematic illustration of an exemplary transparent dressing.

FIG. 7 is a schematic illustration of an exemplary dressing folder.

FIG. 8a is schematic perspective illustration of an inner face of a program of the invention.

FIG. 8b is schematic perspective illustration of an outer face of a program of the invention.

FIG. 9 is a plan view of an inner face of an exemplary SAVG program of the invention.

FIG. 10a is a partial plan view of a third exemplary outer program panel of an outer face of an exemplary SAVG program of the invention.

FIG. 10b is a partial plan view of a first exemplary outer program panel of an outer face of an exemplary SAVG program of the invention.

FIG. 11 is a plan view of an inner face of an exemplary AVF program of the invention.

FIG. 12a is a partial plan view of a third exemplary outer program panel of an outer face of an exemplary AVF program of the invention.

FIG. 12b is a partial plan view of a first exemplary outer program panel of an outer face of an exemplary AVF program of the invention.

FIG. 13 is a plan view of an inner face of an exemplary LAVG program of the invention.

FIG. 14a is a partial plan view of a third exemplary outer program panel of an outer face of an exemplary LAVG program of the invention.

FIG. 14b is a partial plan view of a first exemplary outer program panel of an outer face of an exemplary LAVG program of the invention.

FIG. 15a is a plan view of the front of an exemplary site preparation card.

FIG. 15b is a plan view of the back of an exemplary site preparation card.

FIG. 16 is a schematic illustration of a linear array of mnemonic markers.

FIG. 17 is a schematic illustration of a parallel array of mnemonic markers.

FIG. 18 is a schematic illustration of an image field of exemplary site preparation card containing an exemplary reproduction of an AVF hemodialysis access configuration.

FIG. 19 is a schematic illustration of an image field of exemplary site preparation card containing an exemplary reproduction of an SAVG hemodialysis access configuration.

FIG. 19a is a schematic illustration of the appearance of a reproduction of an AVF hemodialysis configuration just prior to a first hemodialysis session and also shows the appearance of vertically arrayed placeholders just prior to a first hemodialysis session.

FIG. 19b is a schematic illustration of the appearance of a reproduction of an AVF hemodialysis configuration just prior to a second hemodialysis session and also shows the appearance of vertically arrayed placeholders just prior to a second hemodialysis session.

FIG. 19d is a schematic illustration of the appearance of a reproduction of an AVF hemodialysis configuration just prior to a third hemodialysis session and also shows the appearance of vertically arrayed placeholders just prior to a third hemodialysis session.

FIG. 19d is a schematic illustration of the appearance of a reproduction of an AVF hemodialysis configuration just prior to a fourth hemodialysis session and also shows the appearance of vertically arrayed placeholders just prior to a fourth hemodialysis session.

FIG. 19e is a schematic illustration of the appearance of a reproduction of an AVF hemodialysis configuration just prior to a fifth hemodialysis session and also shows the appearance of vertically arrayed placeholders just prior to a fifth hemodialysis session.

FIG. 20 is a schematic illustration of an image field of exemplary site preparation card containing an exemplary reproduction of an LAVG hemodialysis access configuration.

FIG. 20a is a schematic illustration of the appearance of a reproduction of an AVF hemodialysis configuration just prior to a first hemodialysis session and also shows the appearance of vertically arrayed placeholders just prior to a first hemodialysis session.

FIG. 20b is a schematic illustration of the appearance of a reproduction of an LAVG hemodialysis configuration just prior to a second hemodialysis session and also shows the appearance of vertically arrayed placeholders just prior to a second hemodialysis session.

FIG. 20c is a schematic illustration of the appearance of a reproduction of an LAVG hemodialysis configuration just prior to a third hemodialysis session and also shows the appearance of vertically arrayed placeholders just prior to a third hemodialysis session.

FIG. 20d is a schematic illustration of the appearance of a reproduction of an LAVG hemodialysis configuration just prior to a fourth hemodialysis session and also shows the appearance of vertically arrayed placeholders just prior to a fourth hemodialysis session.

FIG. 20e is a schematic illustration of the appearance of a reproduction of an LAVG hemodialysis configuration just prior to a fifth hemodialysis session and also shows the appearance of vertically arrayed placeholders just prior to a fifth hemodialysis session.

FIG. 20f is a schematic illustration of the appearance of a reproduction of an LAVG hemodialysis configuration just prior to a sixth hemodialysis session and also shows the appearance of vertically arrayed placeholders just prior to a sixth hemodialysis session.

### DETAILED DESCRIPTION OF THE INVENTION

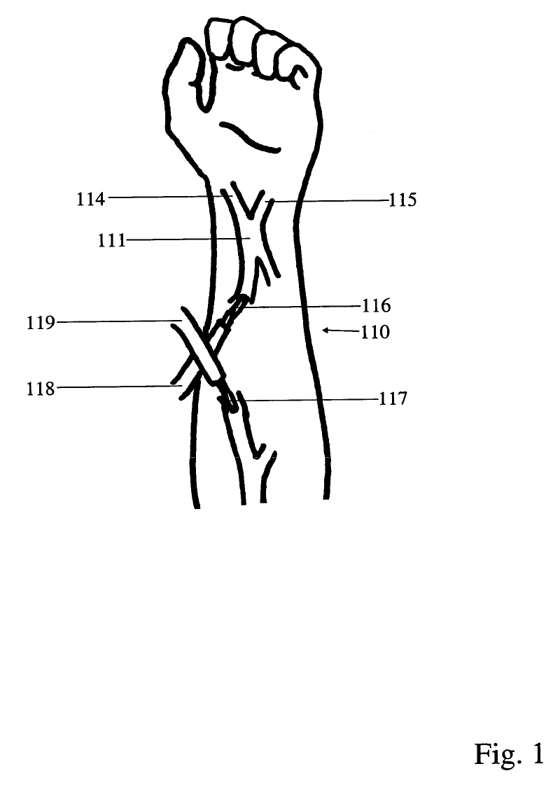

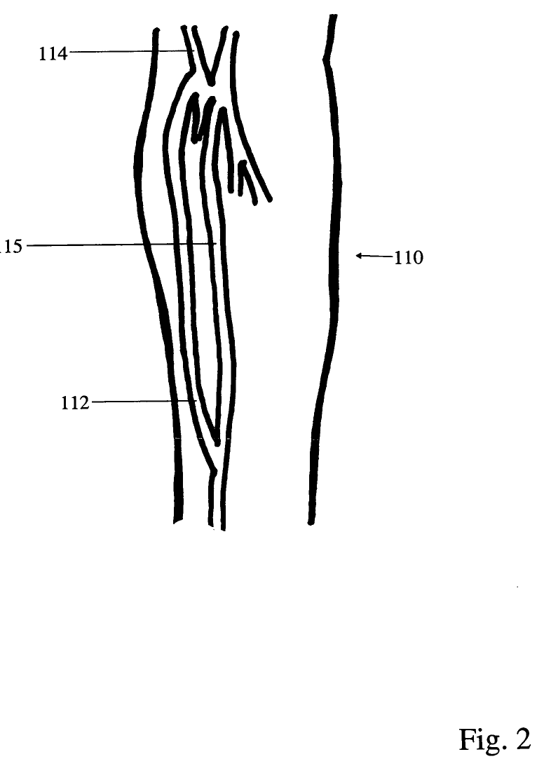

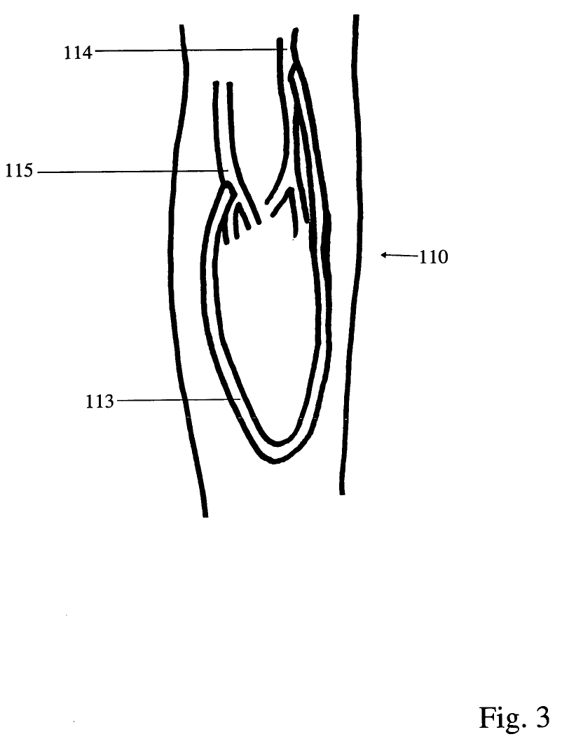

As indicated hereinabove, a patient with ESRD must have a hemodialysis access configuration surgically constructed, along which, at hemodialysis cannulation sites, paired arterial and venous needles are inserted for withdrawing toxin-laden blood and returning freshly-dialyzed blood. Hemodialysis access configurations commonly take the form of an AVF, SAVG or LAVG, which are respectively shown in FIG. 1, FIG. 2 and FIG. 3

In FIG. 1, a hemodialysis access configuration in the form of an AVF 111 is shown in a semi-transparent supinated forearm 110 of a hemodialysis patient. AVF 111 surgically unites forearm vein 114 and forearm artery 115. Efferent hemodialysis cannulation site 116 and afferent hemodialysis cannulation site 117 respectively provide access for efferent flow tubing 118 to a hemodialyzer (not shown) and afferent flow tubing 119 from a hemodialyzer.

In FIG. 2, a hemodialysis access configuration in the form of a SAVG 112 is shown in a semi-transparent supinated forearm 110 of a hemodialysis patient. SAVG 112 forms a straight conduit that unites forearm vein 114 and forearm artery 115.

In FIG. 3, a hemodialysis access configuration in the form of a LAVG 113 is shown in a semi-transparent supinated forearm 110 of a hemodialysis patient. LAVG 113 forms a looped conduit that unites forearm vein 114 and forearm artery 115.

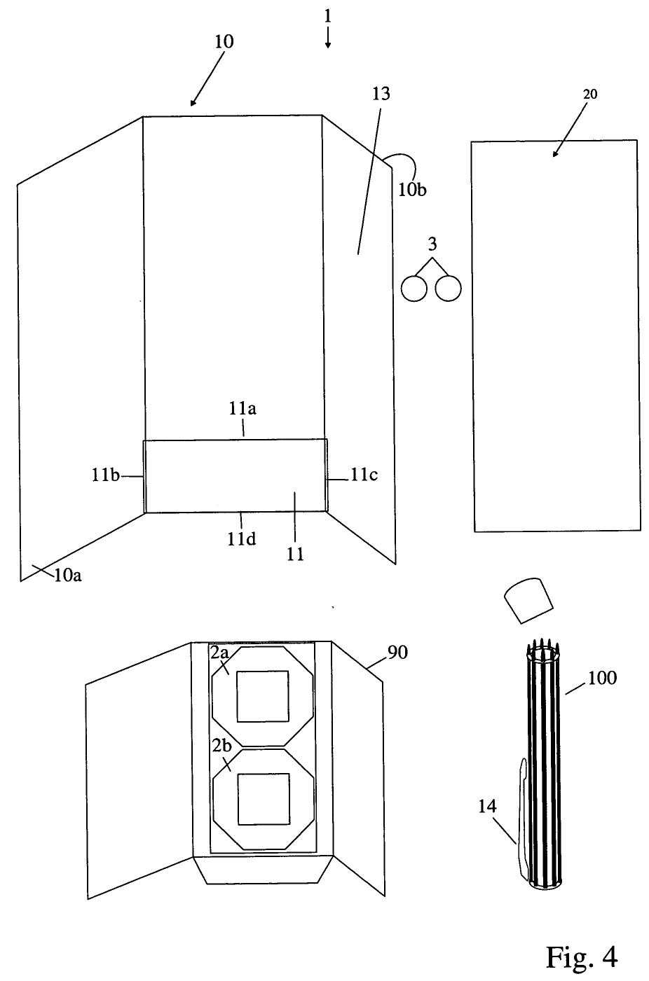

There is shown in FIG. 4 a perspective schematic illustration of a common exemplary embodiment of a device 1 for the rotatable selection, preparation and recollection of hemodialysis cannulation sites along a hemodialysis access configuration of a patient with ESRD, comprising a foldable sheet of material (hereinafter “program”) 10 having an inner face 10a, and an outer face 10b, an exemplary site preparation card 20, two exemplary transparent dressings 2a and 2b encased in an exemplary dressing folder 90, a plurality of exemplary numbered self-adhesive mnemonic markers (“mnemonic markers”) 3, and an exemplary skin marking device 100.

As shown in FIG. 4, inner face 10a of program 10 has means 11 for the removable receipt of exemplary site preparation card 20 and the removable receipt of exemplary transparent dressings 2a and 2b encased in an exemplary dressing folder 90. Inner face 10a of program 10 also has means 13 for the removable receipt of exemplary mnemonic markers 3.

Exemplary skin marking device 100 has means 14 for the removable attachment of exemplary skin marking device 100 to program 10.

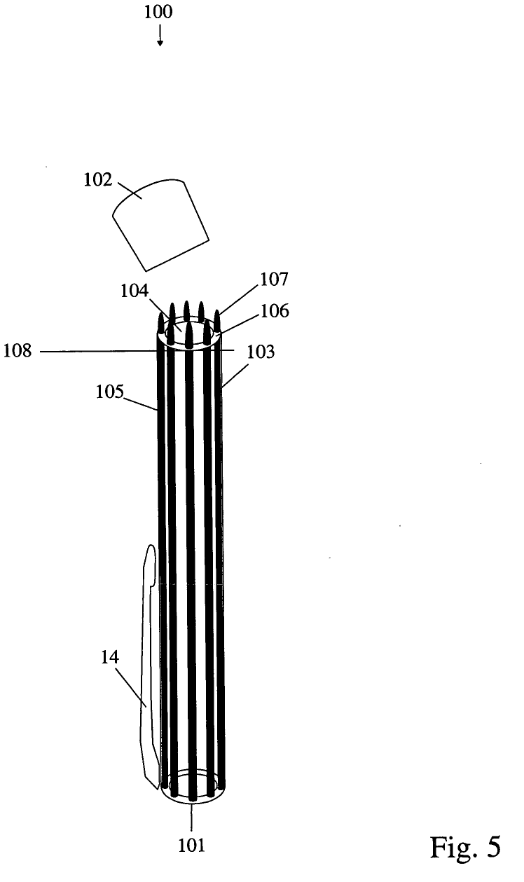

Exemplary skin marking device 100 for use in conjunction with program 10 is shown in greater detail in FIG. 5. Exemplary skin marking device 100 is carried upon or within program 10 by means 14 for the removable attachment of skin marking device 100 to program 10. Exemplary skin marking device 100 is used by an ESRD patient to mark portions of skin overlying a hemodialysis access configuration that have been selected as hemodialysis cannulation sites by the ESRD patient using the invention. About 1 to 2 hours before an anticipated hemodialysis session and following the selection of hemodialysis cannulation sites by methods described hereinbelow, the ESRD patient's actual hemodialysis cannulation sites, corresponding to representative hemodialysis sites selected from an image of the ESRD patient's hemodialysis access configuration appearing on exemplary site preparation card 20, are marked with exemplary marking device 100.

Exemplary skin marking device 100 for use in conjunction with program 10 is sized to be comfortably used as an instrument for making marks by hand, even by those ESRD patients having limited mobility owing to an infirmity of the hands. Exemplary skin marking device 100 effectively functions as a stamp that outlines a circular area of the skin of an ESRD patient merely by pressing skin marking device 100 against the skin.

Exemplary marking device 100 for use in conjunction with program 10 is comprised of a tubular body 101 that is adapted to be fitted by a cap 102. Tubular body 101 is comprised of an outer cylinder 103 concentric about an inner cylinder 104, between which cylinders there is circumferentially disposed an exemplary set of eight markers 105 that are equidistant from one another. Exemplary set of eight markers 105 are secured in annular space 106 between outer cylinder 103 and inner cylinder 104 by nontoxic glue, or any other substantially equivalent adhesive substance. Outer cylinder 103 may be made of metal or plastic, or a substantially equivalent material, and has a radius of about 12/16 inch, a length of about 6 inches and a thickness of about 1/16 inch. Inner cylinder 104 may also be made of metal or plastic, or a substantially equivalent material, and has a radius of about 10/16 inch, a length of about 6 inches and a thickness of about 1/16 inch.

Exemplary set of eight markers 105 may, for example, be comprised of felt-tipped markers, or any other substantially equivalent device or instrument suitable for the carriage of non-permanent, non-toxic ink for marking the skin. Marking tips 107 of exemplary set of eight markers 105 extend beyond top margin 108 of tubular body 101 and, when pressed against the skin of an ESRD patient cause an exemplary eight dots to be marked that define a circle outlining an actual hemodialysis cannulation site selected by the ESRD patient.

There is affixed to outer cylinder 103, means 14 for the removable attachment of exemplary skin marking device 100 to program 10. Means 14 may, for example, comprise a clip, as shown in FIG. 5, or a clasp, clamp or substantially equivalent fastening device or, for example, a strip of material, such as, for example, Velcro®, affixed onto exemplary skin marking device 100 so that exemplary skin marking device 100 may be removably attached to program 10.

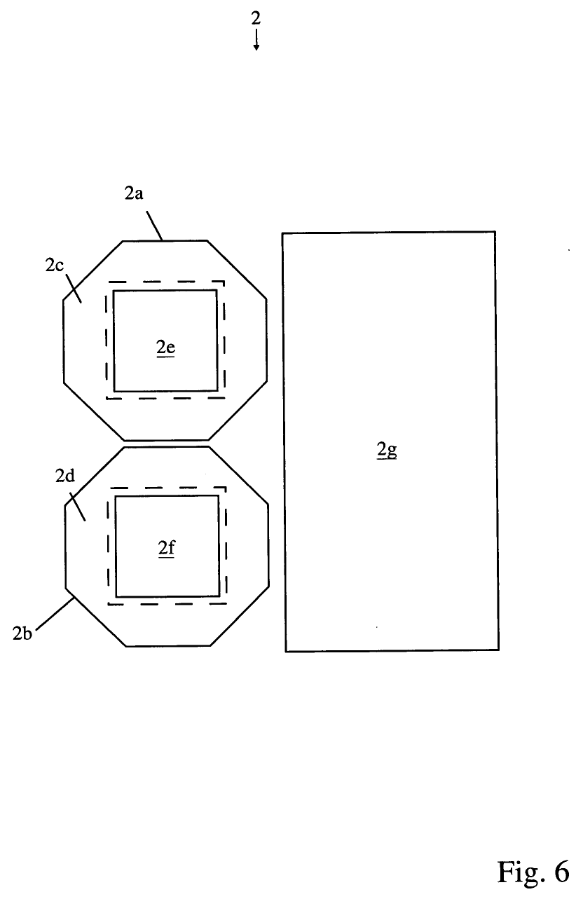

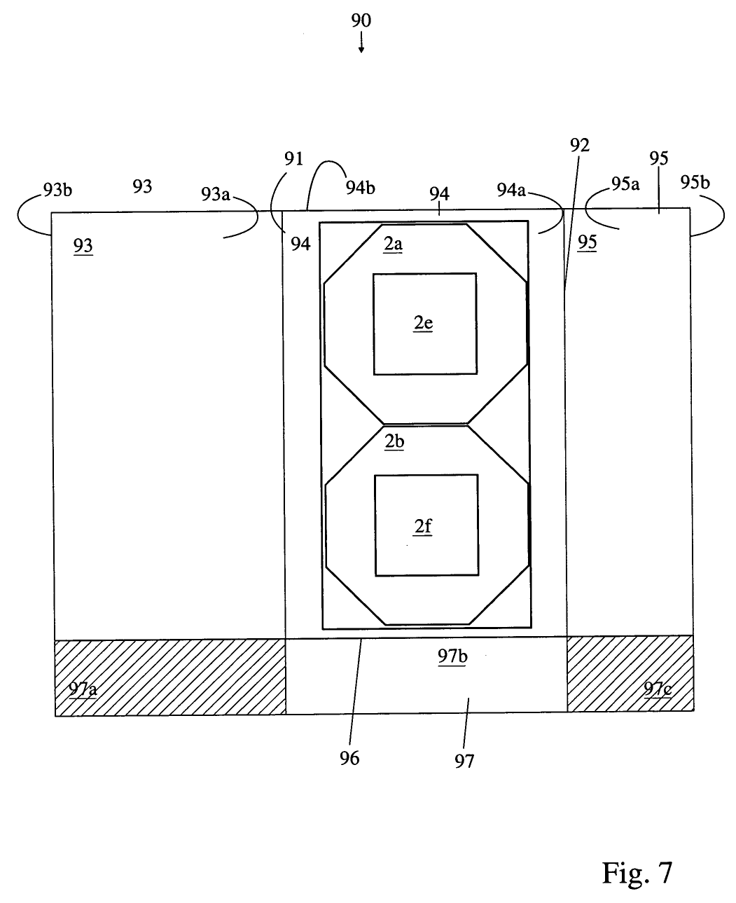

Two exemplary transparent dressings 2a and 2b for use in conjunction with program 10 are shown in FIG. 6. Exemplary transparent dressings 2a and 2b are held in exemplary dressing folder 90, shown in greater detail in FIG. 7, for use in conjunction with program 10. Exemplary dressing folder 90 is also more fully described hereinbelow.

Exemplary transparent dressings 2a and 2b are used by an ESRD patient as a protective coverings over portions of skin that have been selected as actual hemodialysis cannulation sites using the invention. Following the selection of these actual hemodialysis cannulation sites by an ESRD patient using the methods described hereinbelow, and about 1 to 2 hours before an anticipated hemodialysis session, the selected actual hemodialysis cannulation sites, having been marked with exemplary skin marking device 100 (FIG. 4 and FIG. 5), are coated with a dollop of anesthetic ointment, such as, for example, EMLA® cream or an ointment containing lidocaine, prilocaine, or a substantially equivalent anesthetic ingredient. After applying the anesthetic ointment, exemplary transparent dressings 2a and 2b are placed over the skin to prevent smearing and dilution of the anesthetic ointment, and to maintain the anesthetic ointment in contact with the skin of the selected actual hemodialysis cannulation sites.

In FIG. 6, Exemplary transparent dressings 2a and 2b for use in conjunction with program 10 are seen to comprise respective adhesive border portions 2c and 2d contiguously framing respective nonadhesive transparent central windows 2e and 2f, both of which are removably attached to a common backing 2g by an adhesive surface of adhesive border portions 2c and 2d.

Adhesive border portions 2c and 2d may, for example, be made of paper or plastic surgical tape, or a substantially equivalent material having a side adhesive to the skin. Adhesive border portions 2c and 2d may have a width of about 1/2 inch. Each adhesive border portion 2c and 2d is respectively folded over upon itself to form an exemplary octagonal border about each nonadhesive transparent central window 2e and 2f. Transparent central windows 2e and 2f may, for example, be made of cellophane, saran polyvinylidene chloride (PVDC), Mylar®, or a substantially equivalent nonadhesive transparent material, forming a square (dashed lines in FIG. 6) about 1.25 inches on each side, which square is secured to the respective adhesive sides of border portions 2c and 2d which form the octagonal border about respective transparent central portions 2e and 2f.

Common backing 2g be made of paper or plastic, or a substantially equivalent material, in the approximate form of a rectangle having one side about 2 inches long and another side about 4 inches long, to which exemplary transparent dressing 2a and 2b are respectively detachably adhesive. The transparency of each central portion 2e and 2f of respective exemplary transparent dressings 2a and 2b enables an ESRD patient to visualize a dollop of anesthetic ointment as it is being covered with the dressing.

Exemplary dressing folder 90 for use in conjunction with program 10 contains two exemplary transparent dressings 2a and 2b and is shown in FIG. 7. Exemplary dressing folder 90 for use in conjunction with program 10 may, for example, be made of a sheet of paper, plastic, metal foil or a substantially equivalent material. Exemplary dressing folder 90 for use in conjunction with program 10 measures about 6.5 inches in width and about 5 inches in height when it is unfolded. Exemplary dressing folder 90 is carried upon or within program 10 by means 11 for the removable receipt of exemplary dressing folder 90.

Means 11 may comprise a pocket or sleeve or other means for removably receiving exemplary dressing folder 90 upon or within program 10. In FIG. 4, means 11 is shown as an exemplary pocket that holds both exemplary dressing folder 90 and exemplary site preparation card 20 upon or within program 10.

As shown in FIG. 7, the sheet of material used to fashion exemplary dressing folder 90 may be twice folded vertically along a first vertical fold line 91 and a second vertical fold line 92 so as to define a first vertical portion 93, a second vertical portion 94 and a third vertical portion 95 of exemplary dressing folder 90, each vertical portion being hingedly articulated vertically along first vertical fold line 91 and a second vertical fold line 92.

As shown in FIG. 7, the sheet of material used to fashion exemplary dressing folder 90 may also be folded once horizontally along a horizontal fold line 96, to define an inferior horizontal portion 97, having a first segment 97a, that corresponds to first vertical portion 93, a second segment 97b, that corresponds to second vertical portion 94, and a third segment 97c, that corresponds to third vertical portion 95. In fashioning exemplary dressing folder 90, first segment 97a and third segment 97c of inferior horizontal portion 97 are excised (indicated by hatching in FIG. 7), leaving only second segment 97b. Second segment 97b of inferior horizontal portion 97 is hingedly articulated horizontally along horizontal fold line 96.

Vertical portions 93, 94 and 95 have corresponding inner faces 93a, 94a and 95a, and corresponding outer faces 93b, 94b and 95b, upon which textual, graphic, symbolic or iconic information may be printed.

In FIG. 7, two exemplary transparent dressings 2a and 2b are shown. Exemplary transparent dressings 2a and 2b are held against second vertical portion 94 of exemplary dressing folder 90 by first folding second segment 97b of inferior horizontal portion 97, then folding third vertical portion 95 over second vertical portion 94, and finally folding first vertical portion 93 over third vertical portion 95 that has been folded over second vertical portion 94.

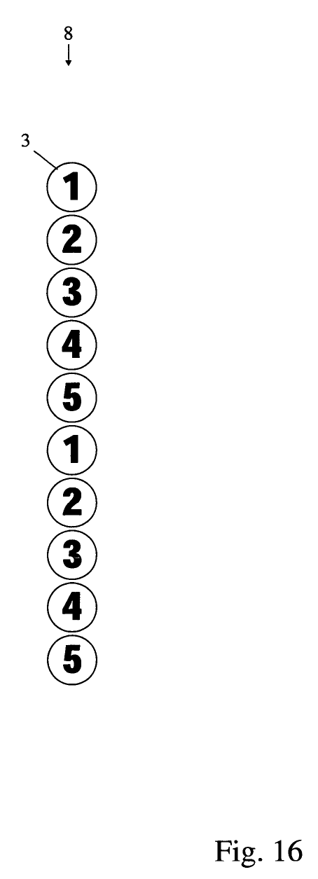

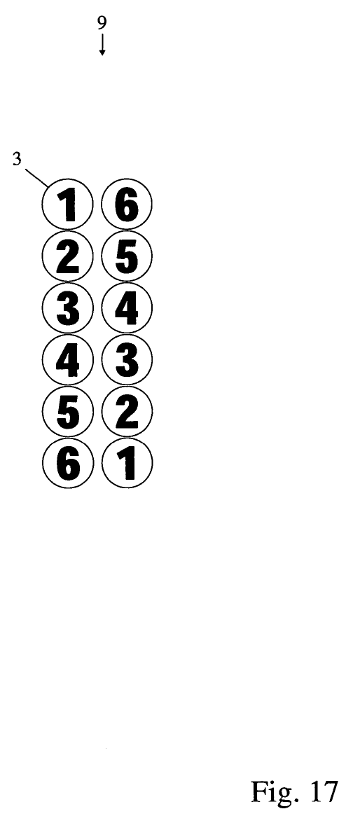

Exemplary mnemonic markers 3 (FIG. 4) for use in conjunction with program 10 are shown in greater detail in FIG. 16 and FIG. 17, and are more fully described hereinbelow.

Exemplary mnemonic markers 3 are carried upon or within program 10 by means 13 (FIG. 4) for the removable receipt of exemplary mnemonic markers 3. Means 13 for the removable receipt exemplary mnemonic markers 3 may comprise a coating, laminate or patch of transparent material upon program 10 to which coating, laminate or patch exemplary mnemonic markers 3 are removably self adhesive.

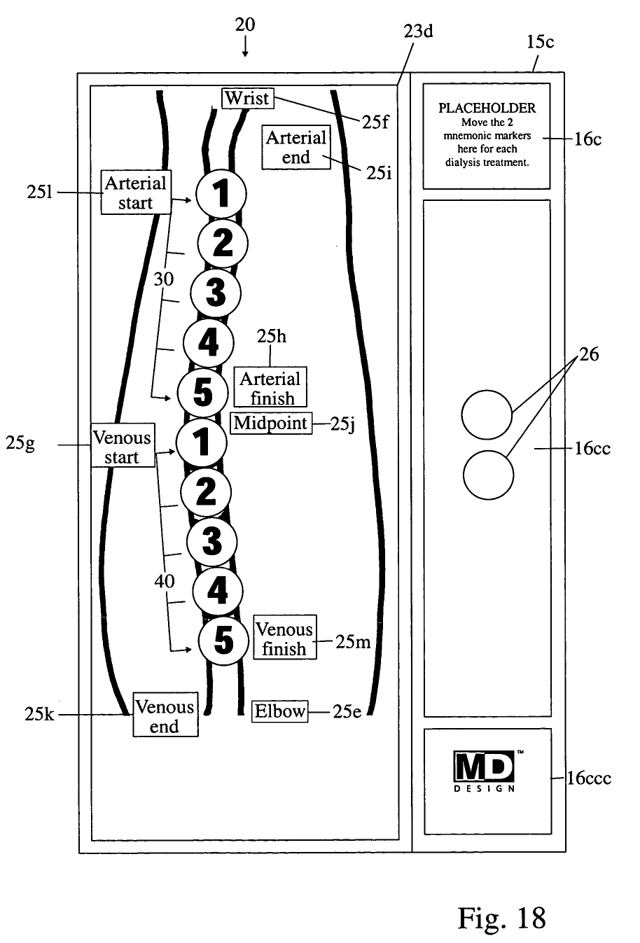

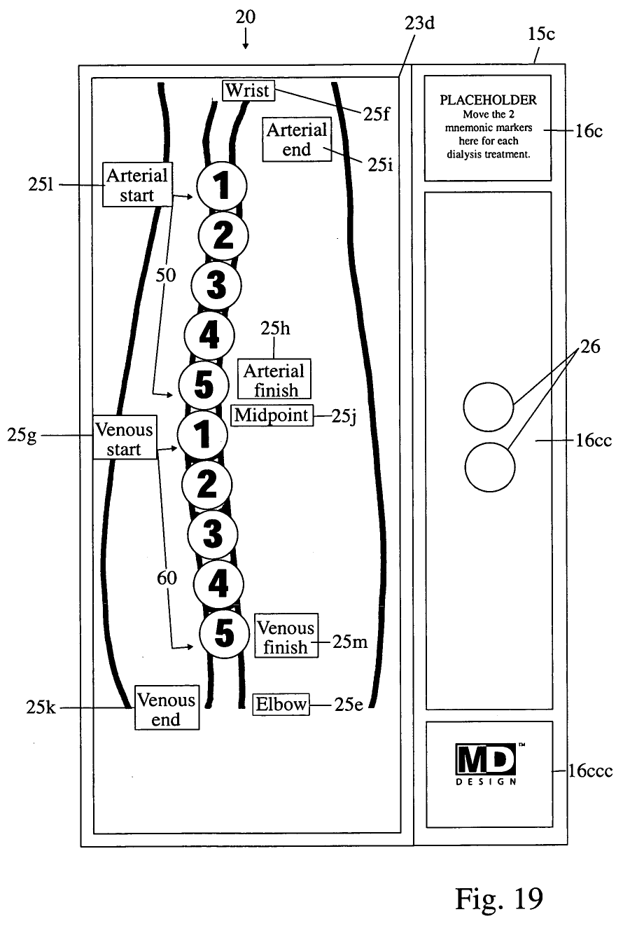

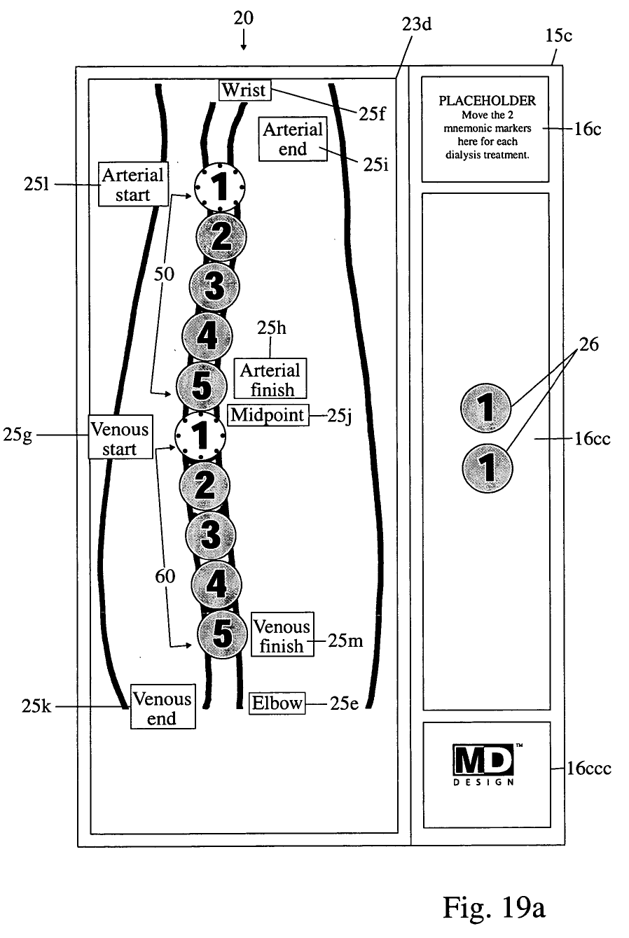

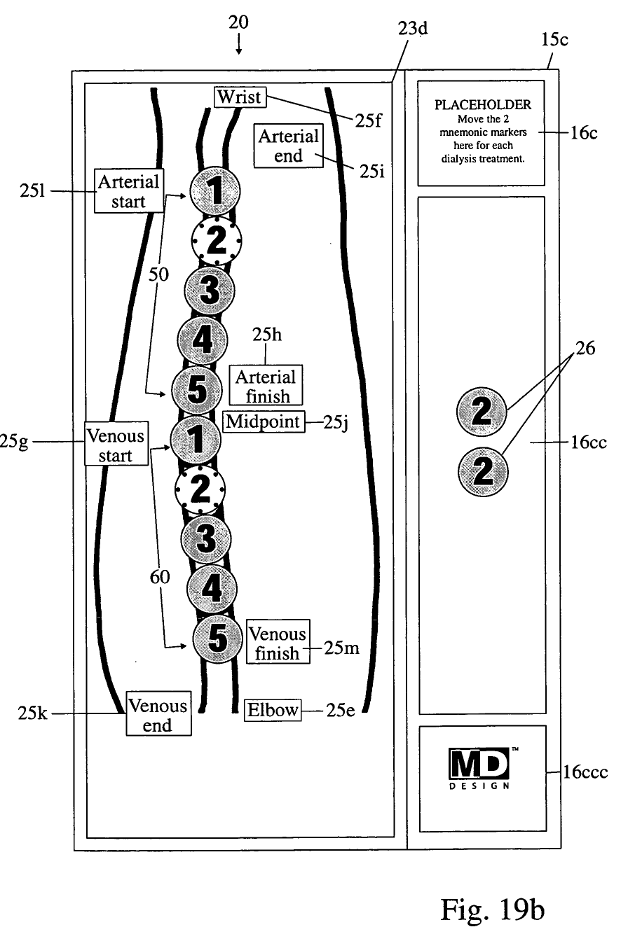

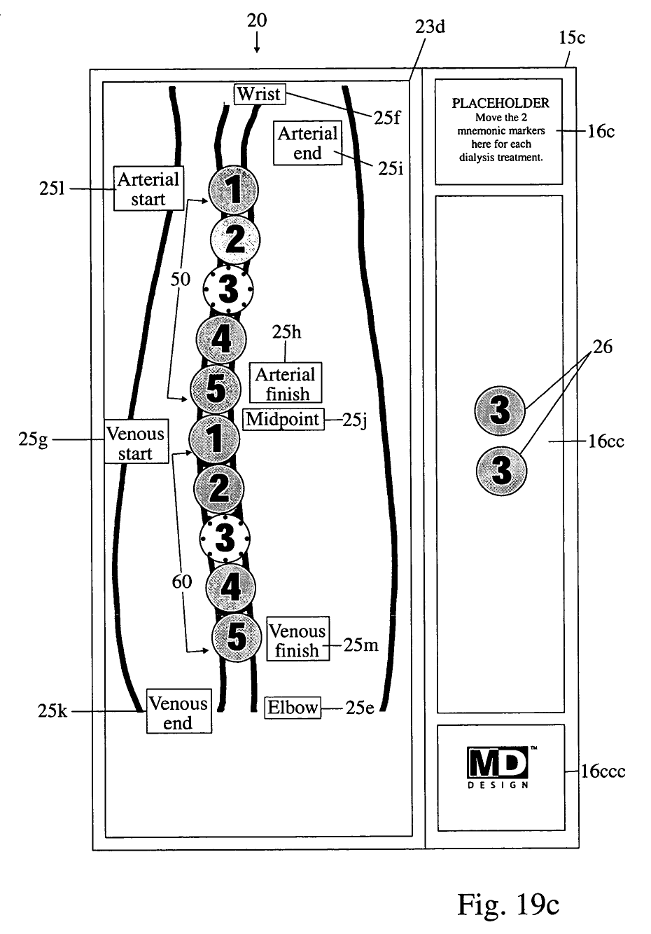

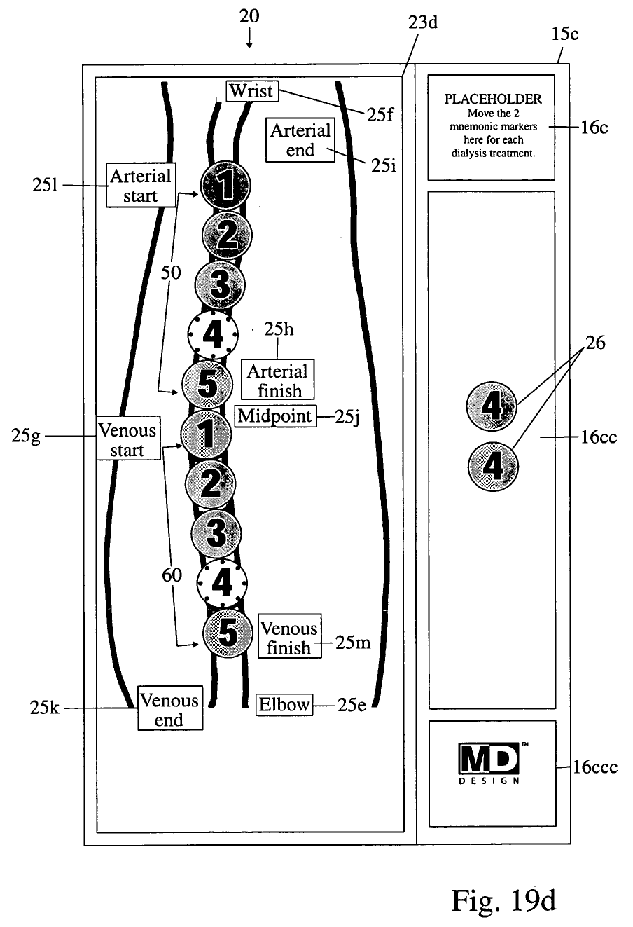

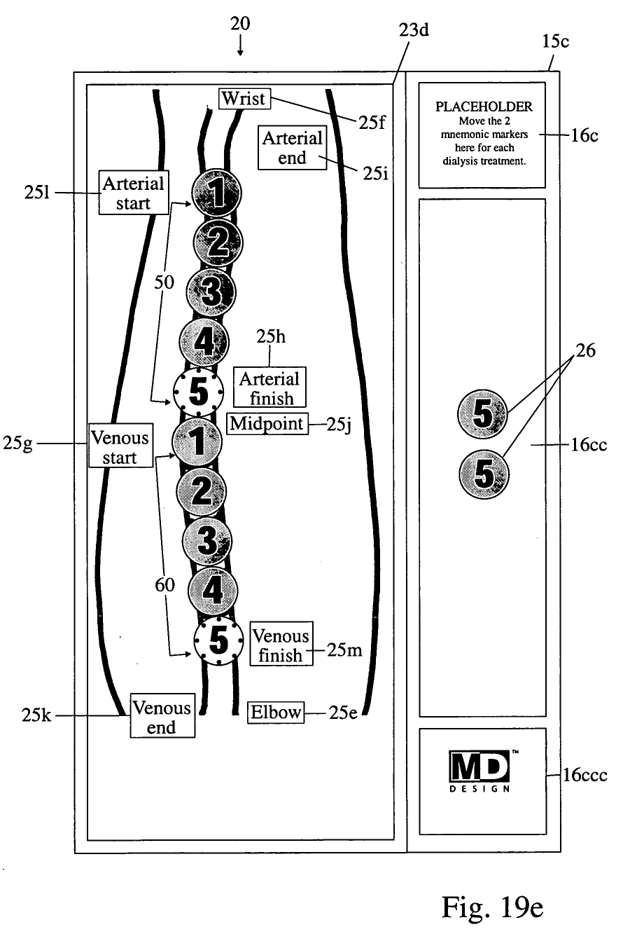

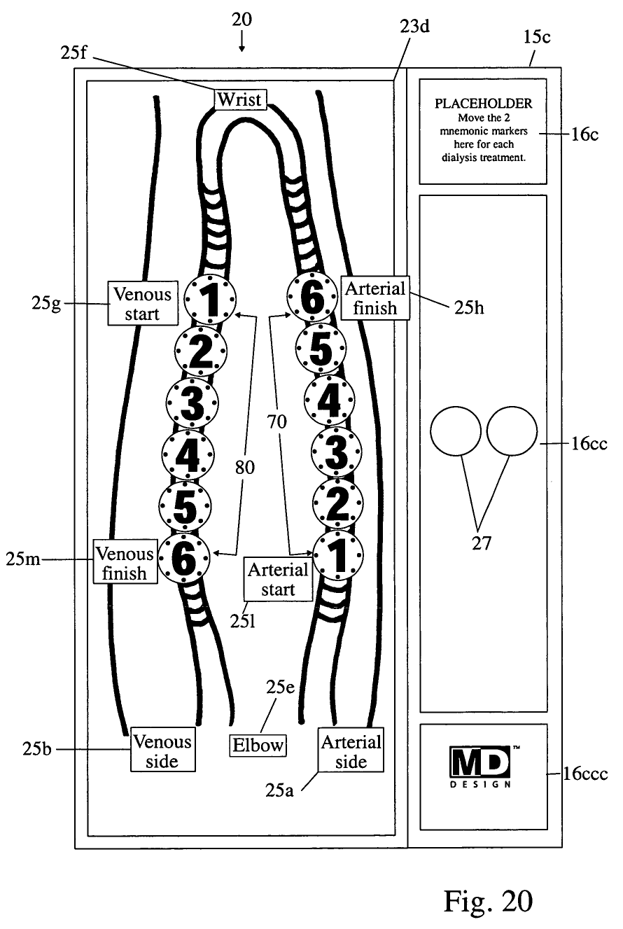

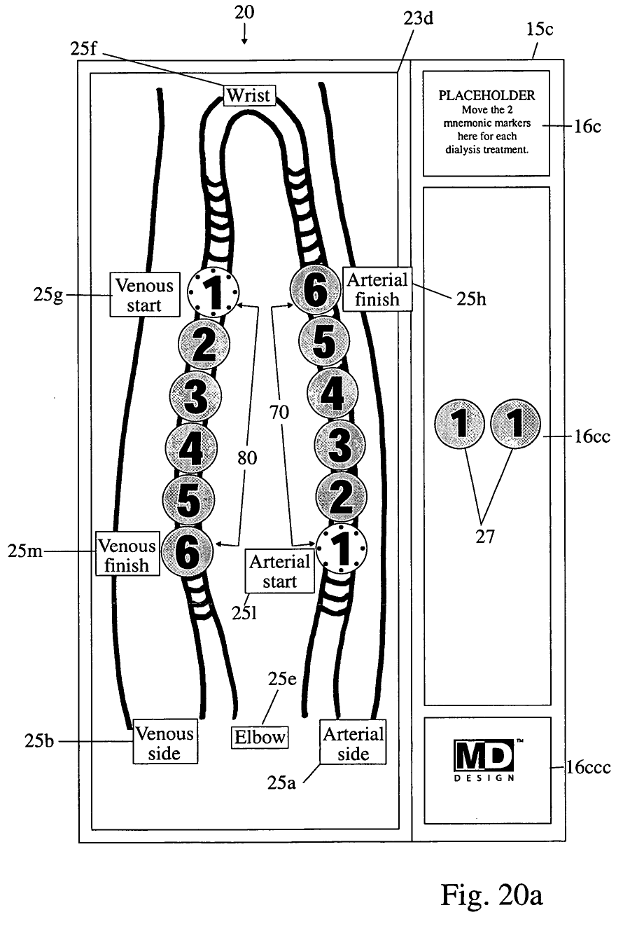

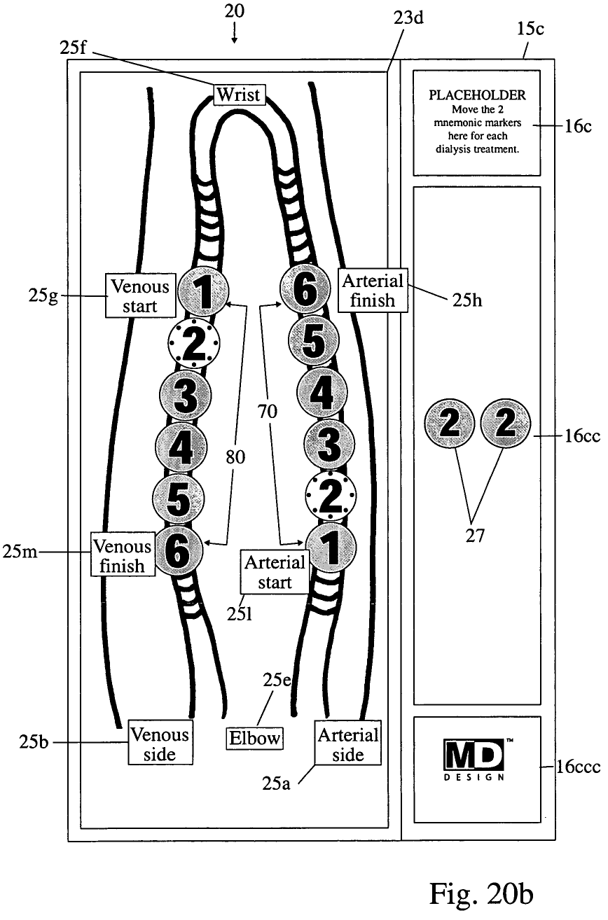

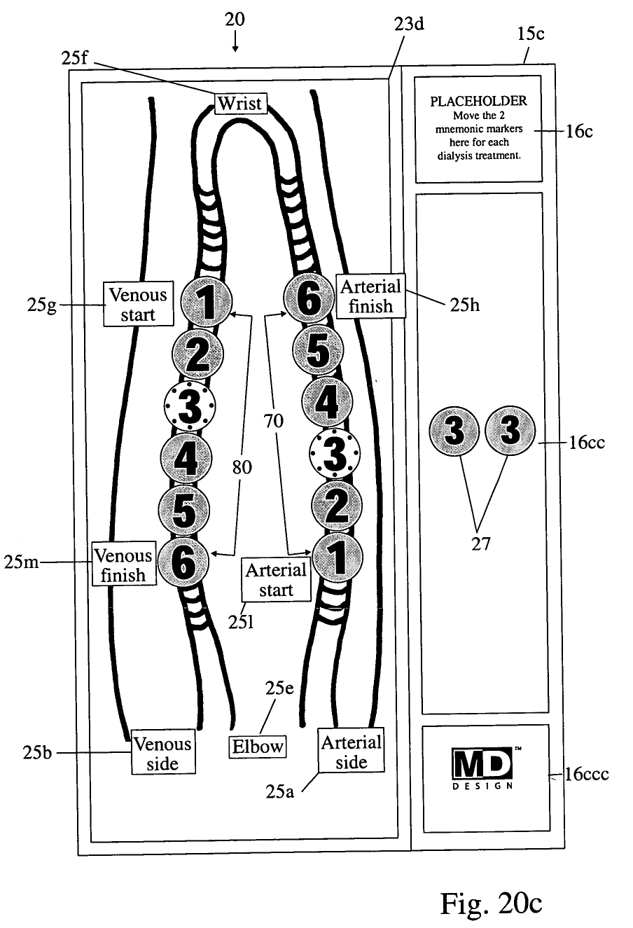

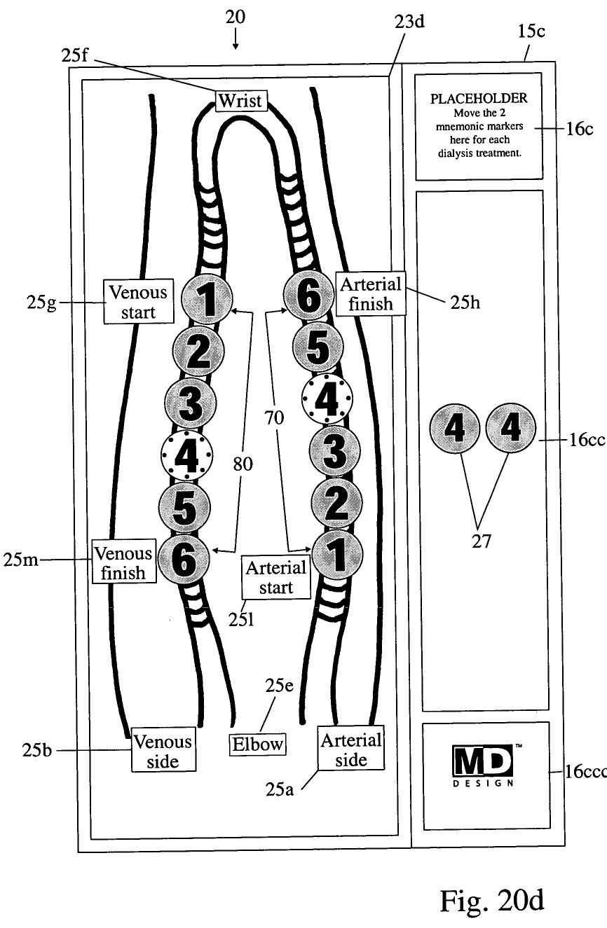

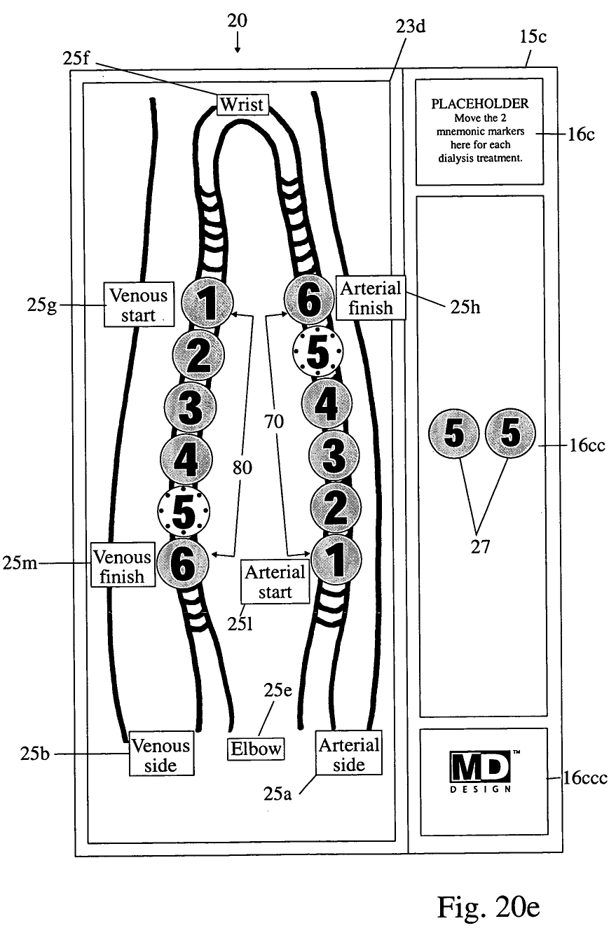

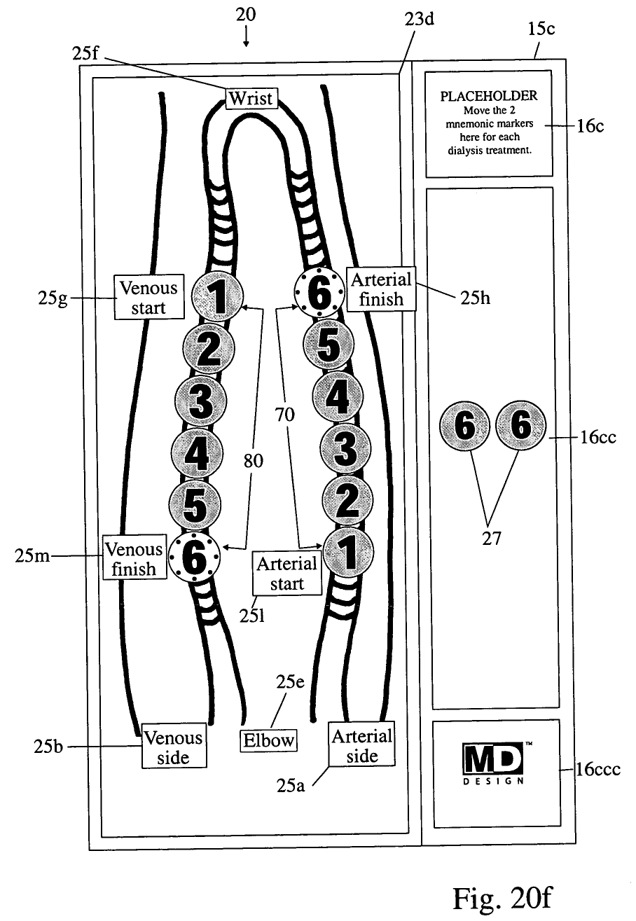

Exemplary site preparation card 20 (FIG. 4) for use in conjunction with program 10 is discussed in greater detail hereinbelow Three exemplary embodiments of exemplary site preparation card 20, corresponding to three exemplary embodiments of the invention, are respectively shown in FIG. 18, FIG. 19 and FIG. 20.

Exemplary site preparation card 20 is carried upon or within program 10 by means 11 (FIG. 4) for the removable receipt of exemplary site preparation card 20. Means 11 may, for example, comprise a sleeve, fold, adhesive strip, slots, or other means for removably receiving exemplary site preparation card 20 upon or within program 10. In FIG. 4, means 11 for the removable receipt of exemplary site preparation card 20 is shown as an exemplary pocket 11a, that is fashioned out of the material of program 10. Exemplary pocket 1a is closed on its lateral sides 11b, 11c, and inferior side 11d, thereby forming an insert space into which exemplary site preparation card 20 and exemplary dressing folder 90 are placed.

Program 10 (FIG. 4) may, for example, be made of paper, plastic, metal foil or a substantially equivalent material. Program 10 is carried on the person of an ESRD patient, and may, for example, measure about 8 inches in height, and about 10 inches in width when fully unfolded

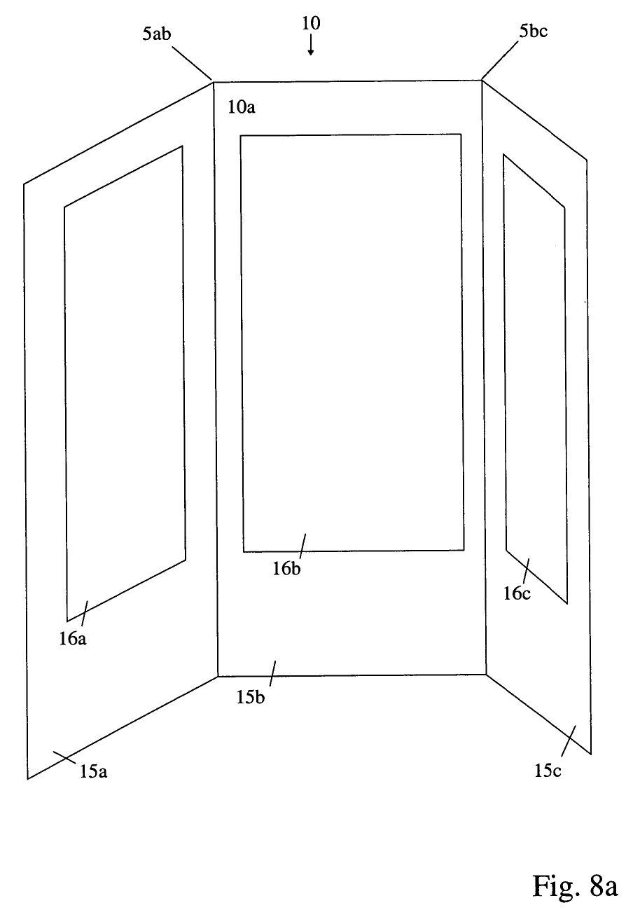

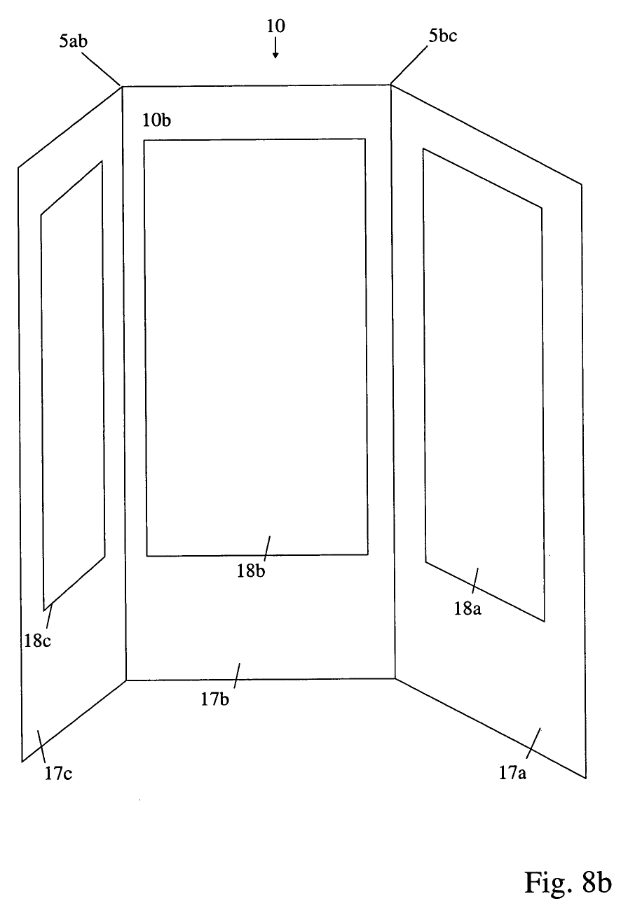

FIG. 8a shows program 10 (FIG. 4) and inner face 10a (FIG. 4) of program 10 in greater detail. In FIG. 8a, program 10 can be seen to be comprised of a set of exemplary program panels 15a, 15b and 15c, respectively denominated as instructional panel 15a, insert panel 15b and placeholder panel 15c. Instructional panel 15a, insert panel 15b and placeholder panel 15c are respectively divided from one another, and may be hingedly articulated from one another, by lines 5ab and 5bc. Lines 5ab and 5bc may, for example, be formed by folds or creases in program 10, or by hinging means, such as, for example, rings or a spiral coil, fashioned into program 10; or, lines 5ab and 5bc may be printed upon inner face 10a of program 10. As shown in FIG. 8a, each exemplary inner program panel 15a, 15b and 15c respectively contains an exemplary inner program field 16a, 16b and 16c, that collectively comprise a set of inner program fields.

FIG. 8b shows outer face 10b (FIG. 4) of a program 10 (FIG. 4) in greater detail. As shown in FIG. 8b, outer face 10b of program 10 is comprised of a set of exemplary outer program panels, 17a, 17b and 17c. As shown in FIG. 8b, each exemplary outer program panel 17a, 17b and 17c respectively contains an exemplary outer program field, 18a, 18b and 18c, comprising a set of outer program fields. In FIG. 8b, exemplary outer program panels, 17a, 17b, and 17c of outer face 10b of program 10 are respectively divided from one another by lines 5ab and 5bc. Exemplary inner program panels 15a, 15b, and 15c shown in FIG. 8a respectively correspond to exemplary outer program panels, 17a, 17b, and 17c, shown in FIG. 8b.

In FIG. 8a, each exemplary inner program field, such as exemplary inner program field 16b, defines a space within an inner program panel, such as insert panel 15b, for the placement of textual, graphic, symbolic or iconic information, or defines a space within an inner program panel, such as insert panel 15b, for the location of the aforementioned means 11 (FIG. 4) for removably receiving exemplary site preparation card 20 and for removably receiving exemplary transparent dressings 2a and 2b encased in exemplary dressing folder 90, or for the location of the aforementioned means 13 (FIG. 4) for removably receiving exemplary mnemonic markers 3.

Each exemplary outer program field, such as exemplary outer program field 18b (FIG. 8b), defines a space within an outer program panel, such as outer program panel 17b (FIG. 8b), for the placement of textual, graphic, symbolic or iconic information.

Three exemplary embodiments of the invention described hereinafter share the common exemplary embodiment of device 1 described hereinabove, shown as an integrated ensemble in FIG. 4, and shown in component detail in FIG. 5, FIG. 6, FIG. 7, FIG. 8a and FIG. 8b. However, each exemplary embodiment of the invention described hereinafter is distinguished by the location, arrangement, content, and use of the program fields contained in the program panels of their respective programs 10, and the content of their corresponding exemplary site preparation cards 20.

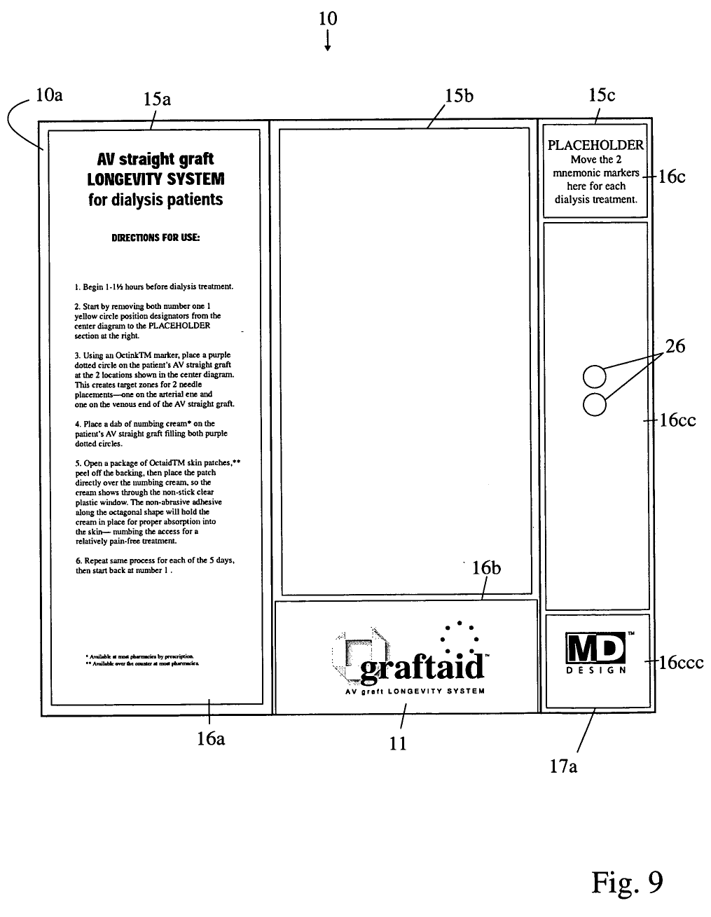

There is shown in FIG. 9 a plan view of inner face 10a of an exemplary SAVG embodiment of program 10 of the invention. In FIG. 9, instructional panel 15a contains one inner program field 16a with exemplary text for instructing an ESRD patient whose hemodialysis access configuration comprises a SAVG on the use of the invention.

In FIG. 9, insert panel 15b contains one inner program field 16b that provides the aforementioned means 11 for removably receiving exemplary site preparation card 20 (FIG. 4 and FIGS. 18-20 infra.) and for removably receiving exemplary transparent dressings 2a and 2b (FIG. 6) encased in exemplary dressing folder 90 (FIG. 7).

In FIG. 9, placeholder panel 15c contains one inner program field 16c holding text instructing an ESRD patient whose hemodialysis access configuration comprises a SAVG on the use of exemplary mnemonic markers 3, another inner program field 16cc (hereinafter “marker placement field”) providing the aforementioned means 13 (FIG. 4) for the removably receiving exemplary mnemonic markers 3 (not shown in FIG. 9), and yet another inner program field 16ccc holding an exemplary manufacturer's logo. In FIG. 9, marker placement field 16cc is shown with two vertically arrayed circular placeholders 26 for the removable receipt of two exemplary mnemonic markers 3 (not shown in FIG. 9).

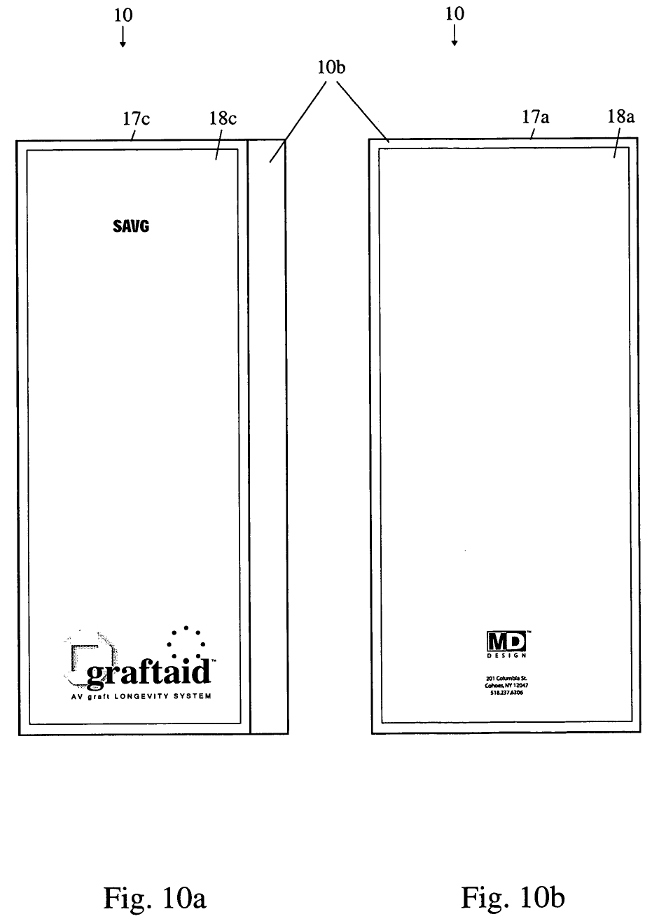

There is shown in FIG. 10a and 10b partial plan views of outer face 10b of an exemplary SAVG embodiment of program 10 of the invention. FIG. 10a shows a plan view of third exemplary outer program panel 17c of outer face 10b of program 10, wherein third exemplary outer program panel 17c is seen to contain third exemplary outer program field 18c, holding both exemplary graphic information and exemplary text identifying program 10 as a program for the SAVG embodiment of the invention. FIG. 10b shows a plan view of first exemplary outer program panel 17a of outer face 10b of program 10, wherein first exemplary outer program panel 17a is seen to contain first exemplary outer program field 18a, holding an exemplary manufacturer's logo and contact information.

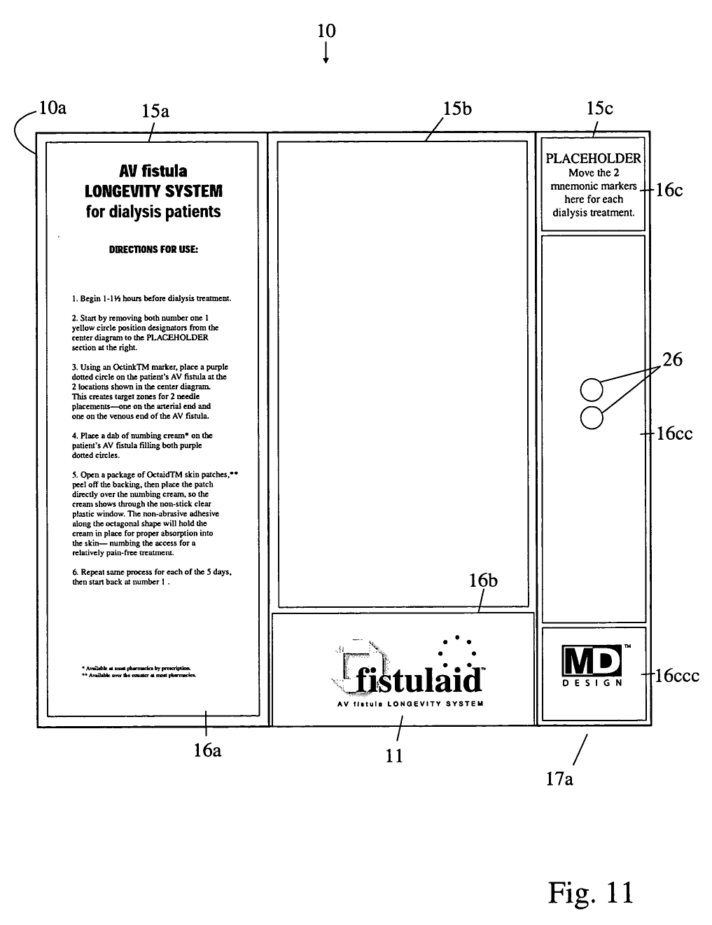

There is shown in FIG. 11 a plan view of inner face 10a of an exemplary AVF embodiment of program 10 of the invention. In FIG. 11, instructional panel 15a contains one inner program field 16a with exemplary text for instructing an ESRD patient whose hemodialysis access configuration comprises an AVF on the use of the invention.

In FIG. 11, insert panel 15b contains one inner program field 16b that provides the aforementioned means 11 for removably receiving exemplary site preparation card 20 (FIG. 4 and FIGS. 18-20 infra.) and for removably receiving exemplary transparent dressings 2a and 2b (FIG. 6) encased in exemplary dressing folder 90 (FIG. 7).

In FIG. 11, placeholder panel 15c contains one inner program field 16c holding text instructing a patient with ESRD whose hemodialysis access configuration comprises an AVF on the use of exemplary mnemonic markers 3, another inner program field 16cc (“marker placement field”) providing the aforementioned means 13 (FIG. 4) for the removable receipt of exemplary mnemonic markers 3 (not shown in FIG. 11), and yet another inner program field 16ccc holding an exemplary manufacturer's logo. In FIG. 11, marker placement field 16cc is shown with two vertically arrayed circular placeholders 26 for the removable receipt of two exemplary mnemonic markers 3 (not shown in FIG. 11).

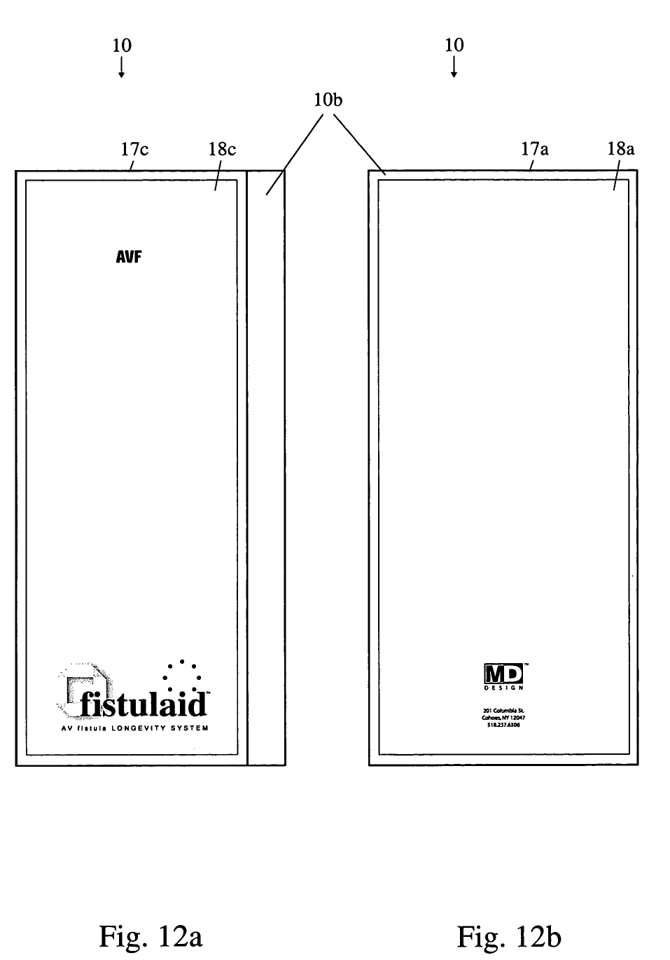

There is shown in FIGS. 12a and 12b partial plan views of outer face 10b (FIG. 4) of an exemplary AVF embodiment of program 10 of the invention. FIG. 12a shows a plan view of third exemplary outer program panel 17c of outer face 10b of program 10, wherein third exemplary outer program panel 17c is seen to contain third exemplary outer program field 18c holding both exemplary graphic information and exemplary text describing program 10 as a program for the AVF embodiment of the invention. FIG. 12b shows a plan view of first exemplary outer program panel 17a of outer face 10b of program 10, wherein first exemplary outer program panel 17a is seen to contain first exemplary outer program field 18a holding an exemplary manufacturer's logo and contact information.

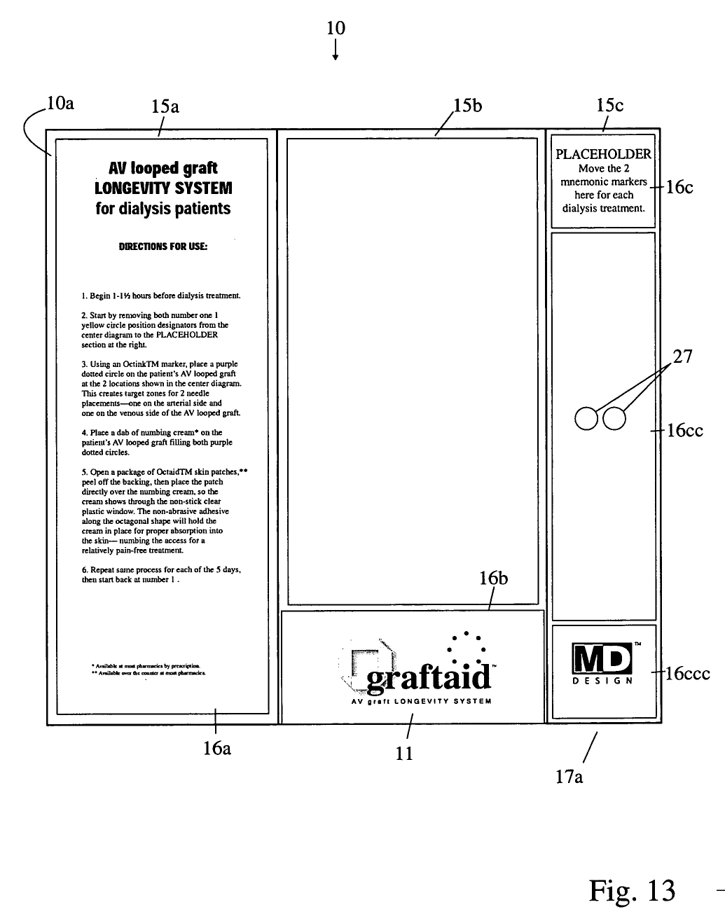

There is shown in FIG. 13 a plan view of inner face 10a an exemplary LAVG embodiment of program 10 of the invention. In FIG. 13, instructional panel 15a contains one inner program field 16a with exemplary text for instructing an ESRD patient whose hemodialysis access configuration comprises a LAVG on the use of the invention.

In FIG. 13, insert panel 15b contains one inner program field 16b that provides the aforementioned means 11 for removably receiving exemplary site preparation card 20 (FIG. 4 and FIGS. 18-20 infra.) and for removably receiving exemplary transparent dressings 2a and 2b (FIG. 6) encased in exemplary dressing folder 90 (FIG. 7).

In FIG. 13, placeholder panel 15c contains one inner program field 16c holding text instructing a patient with ESRD whose hemodialysis access configuration comprises a LAVG on the use of exemplary mnemonic markers 3, another inner program field 16cc (“marker placement field”) providing the aforementioned means 13 for the removable receipt of exemplary mnemonic markers 3 (not shown in FIG. 13), and yet another inner program field 16ccc holding an exemplary manufacturer's logo. In FIG. 13 marker placement field 16cc is shown with two vertically arrayed circular placeholders 27 for the removable receipt of two exemplary mnemonic markers 3 (not shown in FIG. 13).

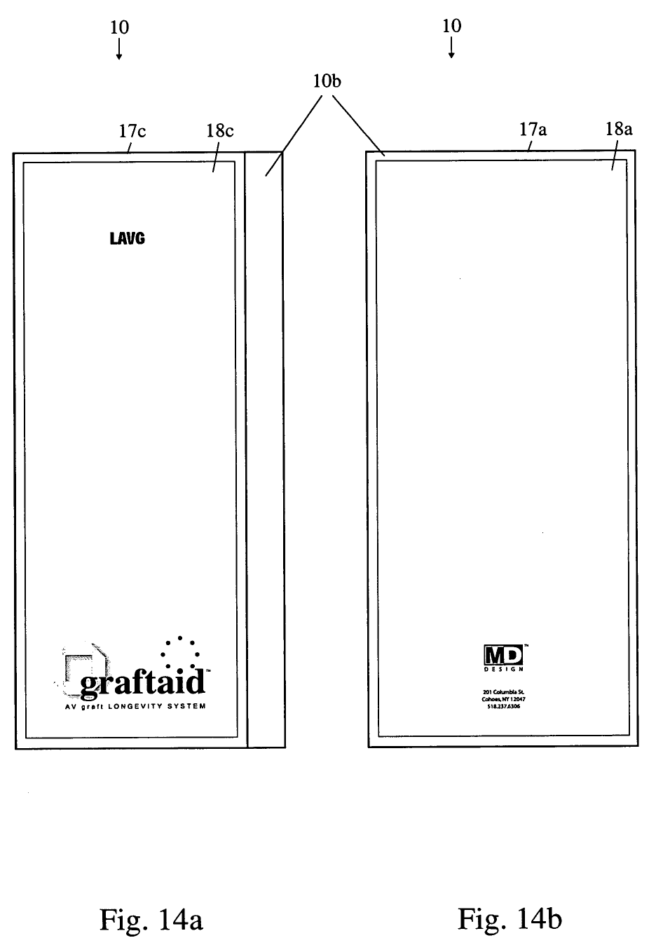

There is shown in FIGS. 14a and 14b partial plan views of outer face 10b an exemplary LAVG embodiment of program 10 of the invention. FIG. 14a shows a plan view of third exemplary outer program panel 17c of outer face 10b of program 10, wherein third exemplary outer program panel 17c is seen to contain third exemplary outer program field 18c holding both exemplary graphic information and exemplary text describing program 10 as the program for the LAVG embodiment of the invention. FIG. 14b shows a plan view of first exemplary outer program panel 17a of outer face 10b of program 10, wherein first exemplary outer program panel 17a is seen to contain first exemplary outer program field 18a holding an exemplary manufacturer's logo and contact information.

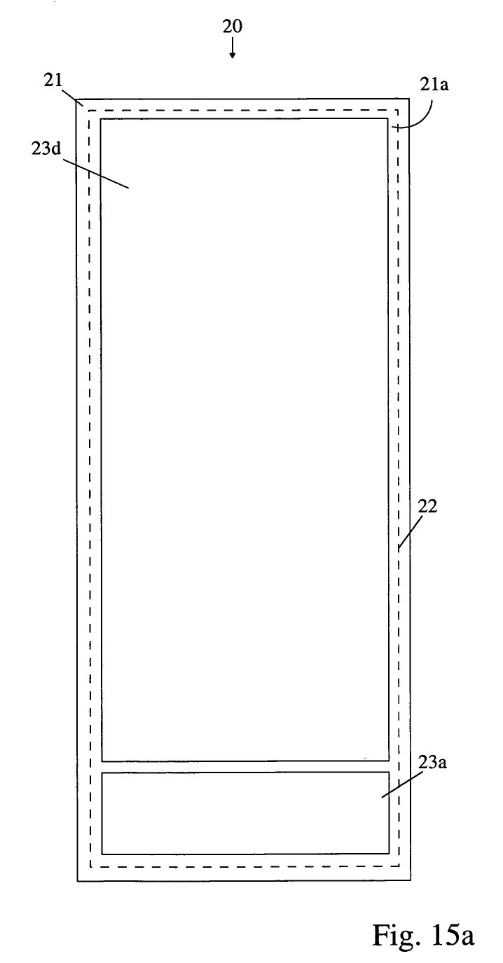

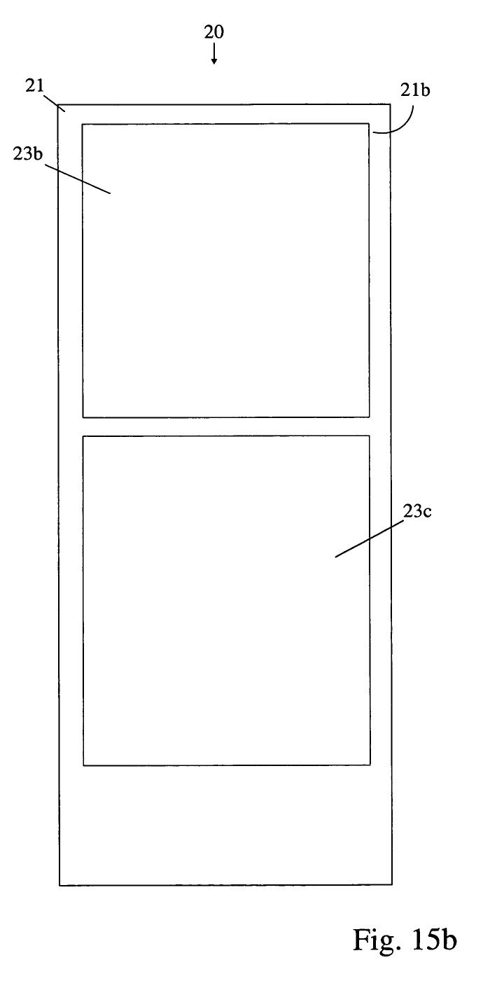

Exemplary site preparation card 20 is shown in greater detail in FIG. 15a and FIG. 15b. In FIG. 15a, exemplary site preparation card 20 is seen to comprise a sheet of card material 21 having a front face 21a. FIG. 15b shows a back face 21b of site preparation card 20. As indicated hereinabove, exemplary site preparation card 20 is removably receivable on or within insert panel 15b (FIG. 8a) of program 10 by the aforementioned means 11 (FIGS. 9, 11 and 13).

Exemplary site preparation card 20 may, for example, also be made of paper, plastic, metal foil or a substantially equivalent material. Exemplary site preparation card 20 may, for example, measure about 8 inches in height and about 3 inches in width.

In FIG. 15a, front face 21a of sheet of card material 21 is laminated with a transparent coating, indicated in FIG. 15a by the area enclosed by dashed lines 22, upon which transparent coating 22 self-adhesive exemplary mnemonic markers 3 (FIGS. 9, 11 and 13) may be detachably and reversibly transferred to and from placeholder inner program panel 15c (FIGS. 9, 11 and 13) of program 10.

In FIG. 15a, front face 21a of sheet of card material 21 contains a set of site preparation fields among which there is included a title site preparation field 23a (“title field”) and an image site preparation field 23d (“image field”). Title field 23a may, for example, contain text identifying exemplary site preparation card 20 as intended for use by a patient with ESRD having an AVF hemodialysis access configuration, a SAVG hemodialysis access configuration or a LAVG hemodialysis access configuration. Image field 23d contains a reproduction of the hemodialysis access configuration of an ESRD patient using the invention, such as, for example, an AVF, a SAVG or a LAVG.

In FIG. 15b, back face 21b of sheet of card material 21 contains a set of site preparation fields among which there is included a patient data site preparation field 23b (“patient field”) and an instruction site preparation field 23c. Patient data field 23b may, for example, contain text identifying the name of the ESRD patient, the name and telephone number of the ESRD patient's health care provider, the address and telephone number of the patient's hemodialysis center, and substantially similar identifying information having medical relevance. Instruction site preparation field 23c may, for example, contain information instructing an ESRD patient on the location, preparation and recollection of a hemodialysis cannulation sites along a hemodialysis access configuration.

The reproduction of the hemodialysis access configuration may be obtained by reproductive means, such as, for example, conventional photography, digital photography, manual drawing, digital imaging or substantially equivalent means. The reproduction of the hemodialysis access configuration may be transferred to image field 23d by transfer means such as, for example, printing, lithography, photocopying, or substantially equivalent means.

FIG. 16 shows an exemplary vertical column 8 (hereinafter “exemplary linear array”) of ten mnemonic markers 3, comprising two sets of five mnemonic markers, bearing numbers 1, 2, 3, 4, 5 on their anterior surfaces. Exemplary linear array of mnemonic markers is used in the rotatable selection, preparation and recollection of hemodialysis cannulation sites along an AVF or SAVG hemodialysis access configuration of an ESRD patient.

FIG. 17 shows two exemplary vertical columns 9 ((hereinafter “exemplary parallel array”) of six exemplary mnemonic markers 3, comprising two sets of six mnemonic markers, each bearing numbers 1, 2, 3, 4, 5, 6 on their anterior surfaces. Exemplary parallel array of mnemonic markers is used in the rotatable selection, preparation and recollection of hemodialysis cannulation sites along a LAVG hemodialysis access configuration of an ESRD patient.

An exemplary mnemonic marker 3 may be of any shape, but is conveniently illustrated in FIG. 16 and FIG. 17 as a circle having a diameter of about 1/2 inch. An exemplary mnemonic marker may be of any color, and may be made of paper, plastic, metallic foil or a substantially equivalent material. The posterior surface of each exemplary mnemonic marker has a quality of “stickiness,” i.e., it is endowed with an impermanent adhesiveness that makes it removably and transferably adhesive to both the surface of image field 23d (FIG. 15a) and marker placement field 16cc contained in placement panel 15c of program 10 (FIGS. 9, 11 and 13). This impermanent adhesiveness may, for example, be secured by an appropriate sticky coating or by static cling that obtains between the posterior surface of an exemplary mnemonic marker 3 and the surfaces of both image field 23d (FIG. 15a) and marker placement field 16cc contained in placement panel 15c of program 10 (FIGS. 9, 11 and 13).

FIG. 18 shows image field 23d of exemplary site preparation card 20 alongside placeholder panel 15c of program 10. In FIG. 18, image field 23d contains an exemplary reproduction of an AVF hemodialysis access configuration. Placeholder panel 15c contains inner program field 16c holding text instructing a patient with ESRD whose hemodialysis access configuration comprises an AVF on the use of exemplary mnemonic markers 3. Marker placement field 16cc providing means 13 (FIG. 4) for the removable receipt of exemplary mnemonic markers 3 (not shown in FIG. 18). Inner program field 16ccc holds an exemplary manufacturer's logo. Marker placement field 16cc is shown with two vertically arrayed circular placeholders 26 for the removable receipt of two exemplary mnemonic markers 3 (not shown in FIG. 18). When image field 23d contains an exemplary reproduction of an AVF (or a SAVG) hemodialysis access configuration, it is used in conjunction with exemplary linear array of mnemonic markers 8 shown in FIG. 16.

When, as shown in FIG. 18, image field 23d contains an exemplary reproduction of an AVF hemodialysis access configuration, a set of image identification fields (identified in FIG. 18 by reference numerals 25e through 25m) is superimposed upon the AVF hemodialysis access configuration. As shown in FIG. 18, the set of image identification fields 25 includes: elbow image identification field 25e that may, for example, may the word “Elbow;” wrist image identification field 25f that may, for example, contain the word “Wrist;” and, Arterial End image identification field 25i that may, for example, contain the words “end of artery,” or the words “Arterial end.” Arterial End image identification field 25i is proximal to wrist image identification field 25f and is associated with Arterial Start image identification field 25l, that may, for example, contain the words “Artery start,” or the words “Arterial start.”

The set of image identification fields further includes Venous End image identification field 25k that may, for example contain the words “Venous end” or “End of vein.” Venous End image identification field 25k is proximal to elbow image identification field 25e, and is also associated with Venous Finish image identification field 25m, that may, for example, contain the words “Vein finish,” or the words “Venous finish.”

The set of image identification fields additionally includes Midpoint image identification field 25j that may, for example contain the word “Midpoint” or the word “Middle.” Midpoint image identification field 25j identifies the approximate anatomic midpoint of an AVF hemodialysis access configuration. Midpoint image identification field 25j is associated with a slightly distal (wrist-ward)

Arterial Finish image identification field 25h that may, for example contain the word “Finish” or the words “Arterial finish.” Midpoint image identification field 25j is also associated with a slightly proximal (elbow-ward) Venous Start image identification field 25g that may, for example contain the word “start” or the words “Venous start.”

As shown in FIG. 18, between Arterial Finish image identification field 25l and Arterial Finish position image identification field 25h, there is superimposed a first approximately linear array of an exemplary five AVF circles 30; and, between Venous Start image identification field 25g and Venous Finish image identification field 25m, there is superimposed a second approximately linear array of an exemplary five AVF circles 40.

The circles of first approximately linear array of an exemplary five AVF circles 30 are numbered in ascending order from 1 to 5, the first such circle being about coincident with Arterial Start image identification field 25l, and the 5th such circle being about coincident with Arterial Finish image identification field 25h. First approximately linear array of an exemplary five AVF circles 30 overlies the expanse of an AVF from Arterial Start image identification field 25l to Arterial Finish image identification field 25h.

The circles of second approximately linear array of an exemplary five AVF circles 40 are also numbered in ascending order from 1 to 5, the first such circle being about coincident with Venous Start image identification field 25g, and the 5th such circle being about coincident with Venous Finish image identification field 25m. Second approximately linear array of an exemplary five AVF circles 40 overlies the expanse of an AVF from Venous Start image identification field 25g to Venous Finish image identification field 25m.

Each numbered circle of first approximately linear array of an exemplary five AVF circles 30 comprises an indicator of an actual site at which an arterial access hemodialysis needle is cannulated. Each correspondingly numbered circle of second approximately linear array of an exemplary five AVF circles 40 comprises an indicator of a corresponding actual site at which a venous access hemodialysis needle is simultaneously cannulated.

Accordingly, the circles of first approximately linear array of an exemplary five AVF circles 30 and second approximately linear array of an exemplary five AVF circles 40 identify successively paired representative arterial and venous cannulation sites along the AVF hemodialysis configuraton and serve as placeholders for correspondingly numbered mnemonic markers of exemplary linear array 8 shown in FIG. 16.

Correspondingly numbered circles of first approximately linear array of an exemplary five AVF circles 30 and second approximately linear array of an exemplary five AVF circles 40 comprise representative arterio-venous cannulation site pairs that are successively employed in an exemplary five successive hemodialysis sessions that comprise an exemplary hemodialysis cycle.

By “marching” the paired arterial and venous hemodialysis needles down the numbered pathway of corresponding circles along the AVF, cannulation trauma to the AVF is evenly distributed over time and repeated cannulation of the same sites is avoided. The longevity of the AVF is accordingly extended.

In the AVF embodiment of the invention:
[i] instructional panel 15a, insert panel 15b and placeholder panel 15c of program 10, shown in FIGS. 9, 11, 13, and inner program fields 16a, 16b and 16c of program 10, also shown in FIGS. 9, 11, 13, are adapted to facilitate the rotatable selection, preparation and recollection of actual hemodialysis cannulation sitess along an AVF hemodialysis access configuration of a patient with ESRD;
[ii] instruction site preparation field 23c of exemplary site preparation card 20, shown in FIG. 15b, is adapted to instruct an ESRD patient having an AVF hemodialysis access configuration to rotatably select, prepare and recall actual hemodialysis cannulation sites along the AVF hemodialysis access configuration;
[iii] image site preparation field 23d of exemplary site preparation card 20, shown in FIG. 15a, is adapted to contain a reproduction of the ESRD patient's AVF hemodialysis access configuration; and
[iv] image identification fields 25e through 25m of image site preparation field 23d on exemplary site preparation card 20, shown in FIG. 18, are adapted to facilitate the rotatable selection, preparation and recollection of actual hemodialysis cannulation sites along the AVF hemodialysis access configuration of the ESRD patient.

FIG. 19 shows image field 23d of exemplary site preparation card 20 alongside placeholder panel 15c of program 10. In FIG. 19, image field 23d contains an exemplary reproduction of a SAVG hemodialysis access configuration. Placeholder panel 15c contains inner program field 16c holding text instructing a patient with ESRD whose hemodialysis access configuration comprises a SAVG on the use of exemplary mnemonic markers 3. Marker placement field 16cc provides means 13 (FIG. 4) for the removable receipt of exemplary mnemonic markers 3 (not shown in FIG. 19). Inner program field 16ccc holding an exemplary manufacturer's logo. Marker placement field 16cc is shown with two vertically arrayed circular placeholders 26 for the removable receipt of two exemplary mnemonic markers 3 (not shown in FIG. 19). When image field 23d contains an exemplary reproduction of a SAVG (or an AVF) hemodialysis access configuration, it is used in conjunction with exemplary linear array of mnemonic markers 8 shown in FIG. 16.

When, as shown in FIG. 19, image field 23d contains an exemplary reproduction of an SAVG hemodialysis access configuration, a set of image identification fields (identified in FIG. 19 by reference numerals 25e through 25m) is superimposed upon the SAVG hemodialysis access configuration. As shown in FIG. 19, the set of image identification fields 25 includes: elbow image identification field 25e that may, for example, may the word “Elbow;” wrist image identification field 25f that may, for example, contain the word “Wrist;” and, Arterial end image identification field 25i that may, for example, contain the words “end of artery,” or the words “Arterial end.” Arterial end image identification field 25i is proximal to wrist image identification field 25f and is associated with Arterial Start image identification field 25l, that may, for example, contain the words “Artery start,” or the words “Arterial start.”

The set of image identification fields further includes Venous end image identification field 25k that may, for example contain the words “Venous end” or “End of vein.” Venous end image identification field 25k is proximal to elbow image identification field 25e, and is also associated with Venous Finish image identification field 25m, that may, for example, contain the words “Vein finish,” or the words “Venous finish.”

The set of image identification fields additionally includes Midpoint image identification field 25j that may, for example contain the word “Midpoint” or the word “Middle.” Midpoint image identification field 25j identifies the approximate anatomic midpoint of an SAVG hemodialysis access configuration. Midpoint image identification field 25j is associated with a slightly distal (wrist-ward) Arterial Finish image identification field 25h that may, for example contain the word “Finish” or the words “Arterial finish.” Midpoint image identification field 25j is also associated with a slightly proximal (elbow-ward) Venous Start image identification field 25g that may, for example contain the word “start” or the words “Venous start.”

As shown in FIG. 19, between Arterial Start image identification field 25l and Arterial Finish image identification field 25h, there is superimposed a first approximately linear array of an exemplary five SAVG circles 50; and, between Venous Start image identification field 25g and Venous Finish image identification field 25m, there is superimposed a second approximately linear array of an exemplary five SAVG circles 60.

The circles of first approximately linear array of an exemplary five SAVG circles 50 are numbered in ascending order from 1 to 5, the first such circle being about coincident with Arterial Start image identification field 25l, and the 5th such circle being about coincident with Arterial Finish image identification field 25h. First approximately linear array of an exemplary five SAVG circles 50 overlies the expanse of an SAVG from Arterial Start image identification field 25l to Arterial Finish image identification field 25h.

The circles of second approximately linear array of an exemplary five SAVG circles 60 are also numbered in ascending order from 1 to 5, the first such circle being about coincident with Venous Start image identification field 25g, and the 5th such circle being about coincident with Venous Finish image identification field 25m. Second approximately linear array of an exemplary five SAVG circles 60 overlies the expanse of an SAVG from Venous Start image identification field 25g to Venous Finish image identification field 25m.

In the SAVG embodiment of the invention:
[i] Instructional panels 15a, insert panel 15b and placeholder panel 15c of program 10, shown in FIGS. 9, 11, 13, and inner program fields 16a, 16b and 16c of program 10, also shown in FIGS. 9, 11, 13 are adapted to facilitate the rotatable selection, preparation and recollection of hemodialysis cannulation sitess along a SAVG hemodialysis access configuration of a patient with ESRD;
[ii] instruction site preparation field 23c of exemplary site preparation card 20, shown in FIG. 15b, is adapted to instruct an ESRD patient having a SAVG hemodialysis access configuration to rotatably select, prepare and recall hemodialysis cannulation sitess along the SAVG hemodialysis access configuration;
[iii] image site preparation field 23d of exemplary site preparation card 20, shown in FIG. 15a, is adapted to contain a reproduction of the ESRD patient's SAVG hemodialysis access configuration; and
[iv] image identification fields 25e through 25m, as shown in FIG. 19 and image site preparation field 23d on exemplary site preparation card 20, shown in FIG. 15a, are adapted to facilitate the rotatable selection, preparation and recollection of hemodialysis cannulation sitess along the SAVG hemodialysis access configuration of the ESRD patient.

Each numbered circle of first approximately linear array of an exemplary five SAVG circles 50 comprises an indicator of an actual site at which an arterial access hemodialysis needle is cannulated. Each correspondingly numbered circle of second approximately linear array of an exemplary five SAVG circles 60 comprises an indicator of a corresponding actual site at which a venous access hemodialysis needle is simultaneously cannulated.

Accordingly, the circles of first approximately linear array of an exemplary five SAVG circles 50 and second approximately linear array of an exemplary five SAVG circles 60 identify successively paired representative arterial and venous cannulation sites along the SAVG hemodialysis configuraton and serve as placeholders for correspondingly numbered mnemonic markers of exemplary linear array 8 shown in FIG. 16.

Correspondingly numbered circles of first approximately linear array of an exemplary five SAVG circles 50 and second approximately linear array of an exemplary five SAVG circles 60 comprise representative arterio-venous cannulation site pairs that are successively employed in an exemplary five successive hemodialysis sessions that comprise an exemplary hemodialysis cycle.

By “marching” the paired arterial and venous hemodialysis needles down the numbered pathway of corresponding circles along the SAVG, cannulation trauma to the SAVG is evenly distributed over time and repeated cannulation of the same sites is avoided. The longevity of the SAVG is accordingly extended.

Inasmuch as a SAVG hemodialysis access configuration surgically substitutes a tube between an artery and a vein for the surgically created fistula of an AVF hemodialysis access configuration, the exemplary reproduction of an AVF hemodialysis access configuration shown in FIG. 18. is equally suitable for the rotatable selection, preparation and recollection of actual hemodialysis cannulation sites along a SAVG hemodialysis access configuration, as shown in FIG. 19. Accordingly the discussion that follows in connection with the use of the AVF embodiment of the invention by an ESRD patient with an AVF hemodialysis access configuration is equally applicable to an ESRD patient with a SAVG hemodialysis access configuration.

When the AVF embodiment of the invention is first given to an ESRD patient having an AVF hemodialysis access configuration, image site preparation field 23d on image exemplary site preparation card 20, already has the exemplary ten mnemonic markers 3 comprising exemplary linear array 8 (shown in FIG. 19) in place within correspondingly numbered circles of first approximately linear array of an exemplary five AVF circles 30, and second approximately linear array of an exemplary five AVF circles 40.

FIG. 19a shows the appearance of an exemplary reproduction of an AVF hemodialysis configuration in image site preparation field 23d of site preparation card 20 just prior to a first hemodialysis session and also shows the appearance of vertically arrayed placeholders 26 in marker placement field 16cc of placeholder panel 15c just prior to a first hemodialysis session.

As shown in FIG. 19a, in advance of the first hemodialysis session of an ESRD patient having an AVF hemodialysis configuration, two paired exemplary mnemonic markers numbered 1 are removed from their correspondingly numbered circles overlying the reproduction of the AVF hemodialysis configuration contained in image site preparation field 23d on image exemplary site preparation card 20, and are placed in the two vertically arrayed placeholders 26 contained in marker placement field 16cc of placeholder panel 15c of program 10. Arterio-venous cannulation in the forthcoming first hemodialysis session then occurs at actual cannulation sites along the ESRD patient's actual AVF hemodialysis configuration that correspond to the exposed circles on the reproduction of the ESRD patient's AVF hemodialysis configuration, from which the paired exemplary mnemonic markers numbered 1 were removed.

FIG. 19b shows the appearance of an exemplary reproduction of an AVF hemodialysis configuration in image site preparation field 23d of site preparation card 20 just prior to a second hemodialysis session and also shows the appearance of vertically arrayed placeholders 26 in marker placement field 16cc of placeholder panel 15c just prior to a second hemodialysis session.

As shown in FIG. 19b, in advance of the next—second—hemodialysis session of an ESRD patient having an AVF hemodialysis configuration, the two paired exemplary mnemonic markers numbered 1 are restored to their original positions in the correspondingly numbered circles overlying the reproduction of the AVF hemodialysis configuration contained in image site preparation field 23d on image exemplary site preparation card 20, and the two paired exemplary mnemonic markers numbered 2 are removed from their correspondingly numbered circles and are placed in the two vertically arrayed placeholders 26 contained in marker placement field 16cc of placeholder panel 15c of program 10. Arterio-venous cannulation in the forthcoming second hemodialysis session then occurs at actual cannulation sites along the ESRD patient's actual AVF hemodialysis configuration that correspond to the exposed circles on the reproduction of the ESRD patient's AVF hemodialysis configuration, from which the paired exemplary mnemonic markers number 2 were removed.

FIGS. 19c through 19e respectively show the appearance of an exemplary reproduction of an AVF hemodialysis configuration in image site preparation field 23d just prior to a third, fourth and fifth hemodialysis session and also shows the appearance of vertically arrayed placeholders 26 in marker placement field 16cc of placeholder panel 15c just prior to a third, fourth and fifth hemodialysis session.

As shown in FIGS. 19c through 19e, in advance of the third, fourth and fifth hemodialysis session of an ESRD patient having an AVF hemodialysis configuration, two paired exemplary mnemonic markers respectively numbered 2, 3, 4 are respectively restored to their original positions in correspondingly numbered circles overlying the reproduction of the AVF hemodialysis configuration contained in image site preparation field 23d on image exemplary site preparation card 20, and the two paired exemplary mnemonic markers repectively numbered 3, 4, 5 are removed from their correspondingly numbered circles and are placed in the two vertically arrayed placeholders 26 contained in marker placement field 16cc of placeholder panel 15c of program 10. Arterio-venous cannulation in the forthcoming third, fourth and fifth hemodialysis session then occurs at cannulation sites along the ESRD patient's actual AVF hemodialysis configuration that correspond to the exposed circles on the reproduction of the ESRD patient's AVF hemodialysis configuration, from which the paired exemplary mnemonic markers numbered 3, 4, 5 were removed.

Generalizing from the foregoing, an index i={N−(N−x)} may be defined for identifying an ith hemodialysis session in a sequence of N hemodialyis sessions comprising an ESRD patient's hemodialysis cycle, where x=1, 2, . . . N. When, for example, x=1 and N=5, the first hemodialysis session of the cycle will by identified by i={5−(5−1)}=1. When, for example, x=2 and N=5, the second hemodialysis session of the cycle will by identified by i={5−(5−2)}=2. When, for example, x=N and N=5, the last hemodialysis session of the cycle will by indentified by i={5−(5−5)}=5.

Following an exemplary ith of N hemodialysis sessions for an ESRD patient having an AVF or SAVG hemodialysis configuration, wherein arterio-venous cannulation occurred at cannulation sites along the ESRD patient's actual AVF or SAVG hemodialysis configuration corresponding to paired exposed circles numbered i, appearing on the reproduction of the ESRD patient's AVF or SAVG hemodialysis configuration, paired exemplary mnemonic markers numbered i are removed from the two horizontally arrayed placeholders 27 contained in marker placement field 16cc of placeholder panel 15c and restored to the paired exposed circles numbered i on the reproduction of the ESRD patient's LAVG hemodialysis configuration. Before the next hemodialysis session, the value of x in i={N−(N−x)} is increased by 1, thereby increasing i by 1 and two paired exemplary mnemonic markers numbered i+1 are removed from their correspondingly numbered circles and are placed in the two horizontally arrayed placeholders 27. During the i+1th hemodialysis session, arterio-venous cannulation occurs at cannulation sites along the ESRD patient's actual AVF or SAVG hemodialysis configuration corresponding to the paired exposed circles numbered i+1, appearing on the reproduction of the ESRD patient's AVF or SAVG hemodialysis configuration. Following the i+1th hemodialysis session, the paired exemplary mnemonic markers numbered i+1 are removed from the two horizontally arrayed placeholders 27 and restored to the paired exposed circles numbered i+1. Then, before the next hemodialysis session, the value of x in i={N−(N−x)} is again increased by 1, thereby increasing i+1 by 1 and two paired exemplary mnemonic markers numbered i+2 are removed from their correspondingly numbered circles and are placed in the two horizontally arrayed placeholders 27, etc.

FIG. 20 shows image field 23d of exemplary site preparation card 20 alongside placeholder panel 15c of program 10. In FIG. 20, image field 23d contains an exemplary reproduction of a LAVG hemodialysis access configuration. Placeholder panel 15c contains inner program field 16c holding text instructing a patient with ESRD whose hemodialysis access configuration comprises a LAVG on the use of exemplary mnemonic markers 3. Marker placement field 16cc providing means 13 (FIG. 4) for the removable receipt of exemplary mnemonic markers 3 (not shown in FIG. 20). Inner program field 16ccc holds an exemplary manufacturer's logo. Marker placement field 16cc is shown with two horizontally arrayed circular placeholders 27 for the removable receipt of two exemplary mnemonic markers 3 (not shown in FIG. 20). When image field 23d contains an exemplary reproduction of a LAVG hemodialysis access configuration, it is used in conjunction with exemplary parallel array of exemplary mnemonic markers 9 shown in FIG. 17.

When image field 23d contains an exemplary reproduction of a LAVG hemodialysis access configuration, a set of image identification fields, identified in FIG. 20 by reference numerals 25a, 25b, 25e, 25f, 25g, 25l, 25m, is superimposed upon the LAVG hemodialysis access configuration.

As shown in FIG. 20, the set of image identification fields includes: Arterial side image identification field 25a that may, for example, contain the words “Arterial side,” or the words “Arterial aspect;” Venous side image identification field 25b that may, for example, contain the words “Venous side,” or the words “Vensous aspect;” Elbow image identification field 25e that may, for example, contain the word “Elbow;” Wrist image identification field 25f that may, for example, contain the word “Wrist;” and, Arterial Start image identification field 25l that may, for example, contain the words “Artery start,” or the words “Arterial start.” Arterial Start image identification field 25l is proximal to Elbow image identification field 25e.

The set of image identification fields further includes Arterial Finish image identification field 25h that may, for example, contain the words “Artery finish,” or the words “Arterial finish.” Arterial Finish image identification field 25h is proximal to Wrist image identification field 25f. The set of image identification fields additionally includes Venous Start image identification field 25g that is proximal to Wrist image identification field 25f, and Venous Finish image identification field 25m that is proximal to Elbow image identification field 25e.

As shown in FIG. 20, between Arterial Start image identification field 25l and Arterial Finish image identification field 25h there is interposed a first approximately linear array of an exemplary six LAVG circles 70; and, between Venous Start image identification field 25g and Venous Finish image identification field 25m there is interposed a second approximately linear array of an exemplary six LAVG circles 80.

From the elbow to the wrist, the circles of first approximately linear array of an exemplary six LAVG circles 70 are numbered in ascending order from 1 to 6, the first such circle being about coincident with Arterial Start image identification field 25l, and the sixth such circle being about coincident with Arterial Finish image identification field 25m. First approximately linear array of an exemplary six LAVG circles 70 overlies the expanse of the arterial aspect of a LAVG from Arterial Start image identification field 25l to Arterial Finish image identification field 25h.

From the wrist to the elbow, the circles of second approximately linear array of an exemplary six LAVG circles 80 are numbered in ascending order from 1 to 6, the first such circle being about coincident with Venous Start image identification field 25g, and the sixth such circle being about coincident with Venous Finish image identification field 25m. Second approximately linear array of an exemplary six LAVG circles 80 overlies the expanse of the venous aspect of a LAVG from Venous Start image identification field 25g to Venous Finish image identification field 25m.

In the LAVG embodiment of the invention:
[i] Instructional panels 15a, insert panel 15b and placeholder panel 15c of program 10, shown in FIGS. 9, 11, 13, and inner program fields 16a, 16b and 16c of program 10, also shown in FIGS. 9, 11, 13, are adapted to facilitate the rotatable selection, preparation and recollection of hemodialysis cannulation sitess along a LAVG hemodialysis access configuration of a patient with ESRD;
[ii] instruction site preparation field 23c of exemplary site preparation card 20, shown in FIG. 15b is adapted to instruct an ESRD patient having a LAVG hemodialysis access configuration to rotatably select, prepare and recall hemodialysis cannulation sitess along the LAVG hemodialysis access configuration;
[iii] image site preparation field 23d of exemplary site preparation card 20, shown in FIG. 15a is adapted to contain a reproduction of the ESRD patient's LAVG hemodialysis access configuration; and
[iv] image identification fields 25a, 25b, 25e, 25f, 25g, 25l, 25m, of image site preparation field 23d on exemplary site preparation card 20, shown in FIG. 20, are adapted to facilitate the rotatable selection, preparation and recollection of hemodialysis cannulation sites along the LAVG hemodialysis access configuration of the ESRD patient.

Each numbered circle of first approximately linear array of an exemplary six LAVG circles 70 comprises an indicator of an actual site at which an arterial access hemodialysis needle is cannulated. Each correspondingly numbered circle of second approximately linear array of an exemplary six LAVG circles 80 comprises an indicator of a corresponding actual site at which a venous access hemodialysis needle is simultaneously cannulated.

Accordingly, the circles of first approximately linear array of an exemplary six LAVG circles 70 and second approximately linear array of an exemplary six LAVG circles 80 identify successively paired representative arterial and venous cannulation sites along the LAVG hemodialysis configuraton and serve as placeholders for correspondingly numbered mnemonic markers of exemplary parallel array 9 shown in FIG. 17.

Correspondingly numbered circles of first approximately linear array of an exemplary six LAVG circles 70 and second approximately linear array of an exemplary six LAVG circles 90 comprise representative arterio-venous cannulation site pairs that are successively employed in an exemplary six successive hemodialysis sessions that comprise an exemplary hemodialysis cycle.

By “marching” the paired arterial and venous hemodialysis needles down the numbered pathway of corresponding circles along the LAVG, cannulation trauma to the LAVG is evenly distributed over time and repeated cannulation of the same sites is avoided. The longevity of the LAVG is accordingly extended.

When the LAVG embodiment of the invention is first given to an ESRD patient having an LAVG hemodialysis access configuration, image site preparation field 23d on image exemplary site preparation card 20, already has exemplary twelve mnemonic markers 3 comprising exemplary parallel array 9, shown in FIG. 17, in place within correspondingly numbered circles of first approximately linear array of an exemplary six LAVG circles 70, and second approximately linear array of an exemplary six LAVG circles 80.

FIG. 20a shows the appearance of an exemplary reproduction of an LAVG hemodialysis configuration in image site preparation field 23d just prior to a first hemodialysis session and also shows the appearance of horizontally arrayed placeholders 27 in marker placement field 16cc of placeholder inner program panel 15c just prior to a first hemodialysis session.

As shown in FIG. 20a, in advance of the first hemodialysis session of an ESRD patient having an LAVG hemodialysis configuration, two paired exemplary mnemonic markers numbered 1 are removed from their correspondingly numbered circles overlying the reproduction of the LAVG hemodialysis configuration contained in image site preparation field 23d on image exemplary site preparation card 20, and are placed in the two horizontally arrayed placeholders 27 in marker placement field 16cc of placeholder inner program panel 15c.

Arterio-venous cannulation in the forthcoming first hemodialysis session then occurs at cannulation sites along the ESRD patient's actual LAVG hemodialysis configuration that correspond to the exposed circles on the reproduction of the ESRD patient's LAVG hemodialysis configuration, from which the paired exemplary mnemonic markers numbered 1 were removed.

FIG. 20b shows the appearance of an exemplary reproduction of an LAVG hemodialysis configuration in image site preparation field 23d just prior to a second hemodialysis session and also shows the appearance of horizontally arrayed placeholders 27 in marker placement field 16cc of placeholder inner program panel 15c just prior to a second hemodialysis session.

As shown in FIG. 20b, in advance of the second hemodialysis session of an ESRD patient having an LAVG hemodialysis configuration, the two paired exemplary mnemonic markers numbered 1 are restored to their original positions in the correspondingly numbered circles overlying the reproduction of the LAVG hemodialysis configuration contained in image site preparation field 23d on image exemplary site preparation card 20, and the two paired exemplary mnemonic markers numbered 2 are removed from their correspondingly numbered circles and are placed in the two horizontally arrayed placeholders 27 contained in marker placement field 16cc of placeholder inner program panel 15c.

Arterio-venous cannulation in the forthcoming second hemodialysis session then occurs at cannulation sites along the ESRD patient's actual LAVG hemodialysis configuration that correspond to the exposed circles on the reproduction of the ESRD patient's LAVG hemodialysis configuration, from which the paired exemplary mnemonic markers number 2 were removed.

FIGS. 20c through 20f respectively show the appearance of an exemplary reproduction of an LAVG hemodialysis configuration in image site preparation field 23d just prior to a third, fourth, fifth and sixth hemodialysis session and also shows the appearance of horizontally arrayed placeholders 27 in marker placement field 16cc of placeholder inner program panel 15c, just prior to a third, fourth, fifth and sixth hemodialysis session.

As shown in FIGS. 20c through 20f, in advance of the third, fourth, fifth and sixth hemodialysis session of an ESRD patient having an LAVG hemodialysis configuration, two paired exemplary mnemonic markers respectively numbered 2, 3, 4, 5 are respectively restored to their original positions in correspondingly numbered circles overlying the reproduction of the LAVG hemodialysis configuration contained in image site preparation field 23d on image exemplary site preparation card 20, and the two paired exemplary mnemonic markers respectively numbered 3, 4, 5, 6 are removed from their correspondingly numbered circles and are placed in the two horizontally arrayed placeholders 27 contained in marker placement field 16cc of placeholder panel 15c of program 10 (FIGS. 20c through 20f).

Arterio-venous cannulation in the forthcoming third, fourth, fifth and sixth hemodialysis session then occurs at cannulation sites along the ESRD patient's actual LAVG hemodialysis configuration that correspond to the exposed circles on the reproduction of the ESRD patient's LAVG hemodialysis configuration, from which the paired exemplary mnemonic markers numbered 3, 4, 5, 6 were removed.

Generalizing from the foregoing, an index i={N−(N−x)} may be defined for identifying an ith hemodialysis session in a sequence of N hemodialyis sessions comprising an ESRD patient's hemodialysis cycle, where x=1, 2, . . . N. When, for example, x=1 and N=6, the first hemodialysis session of the cycle will by identified by i={6−(6−1)}=1. When, for example, x=2 and N=6, the second hemodialysis session of the cycle will by identified by i={6−(6−2)}=2. When, for example, x=N and N=6, the last hemodialysis session of the cycle will by indentified by i={6−(6−6)}=6.

Following an exemplary ith of N hemodialysis sessions for an ESRD patient having an LAVG hemodialysis configuration, wherein arterio-venous cannulation occurred at cannulation sites along the ESRD patient's actual LAVG hemodialysis configuration corresponding to paired exposed circles numbered i, appearing on the reproduction of the ESRD patient's LAVG hemodialysis configuration, paired exemplary mnemonic markers numbered i are removed from the two horizontally arrayed placeholders 27 contained in marker placement field 16cc of placeholder panel 15c and restored to the paired exposed circles numbered i on the reproduction of the ESRD patient's LAVG hemodialysis configuration. Before the next hemodialysis session, the value of x in i={N−(N−x)} is increased by 1, thereby increasing i by 1 and two paired exemplary mnemonic markers numbered i+1 are removed from their correspondingly numbered circles and are placed in the two horizontally arrayed placeholders 27. During the i+1th hemodialysis session, arterio-venous cannulation occurs at cannulation sites along the ESRD patient's actual LAVG hemodialysis configuration corresponding to the paired exposed circles numbered i+1, appearing on the reproduction of the ESRD patient's LAVG hemodialysis configuration. Following the i+1th hemodialysis session, the paired exemplary mnemonic markers numbered i+1 are removed from the two horizontally arrayed placeholders 27 and restored to the paired exposed circles numbered i+1. Then, before the next hemodialysis session, the value of x in i={N−(N−x)} is again increased by 1, thereby increasing i+1 by 1 and two paired exemplary mnemonic markers numbered i+2 are removed from their correspondingly numbered circles and are placed in the two horizontally arrayed placeholders 27, etc.

1. A device for the rotatable selection, preparation and recollection of actual hemodialysis cannulation sites along a hemodialysis access configuration of an ESRD patient comprising: a program having an inner face and an outer face; a site preparation card; at least two transparent dressings held in a dressing folder; at least two transferably detachable self-adhesive mnemonic markers; and, a skin marking device having means for removably attaching said skin marking device to said program, wherein said inner face of said program has means for removably receiving said site preparation card and for removably receiving said at least two transparent dressings held in said dressing folder, and means for removably receiving said at least two transferably detachable self-adhesive mnemonic markers.

2. The device of claim 1, wherein said skin marking device comprises a tubular body adapted to receive a cap, said tubular body being formed of an outer cylinder, to which outer cylinder there is affixed said means for removably attaching said skin marking device to said program, said outer cylinder being concentric with an inner cylinder, and between which cylinders there is circumferentially disposed a set of markers whose tips extend beyond a top margin of said tubular body.

3. The device of claim 1, wherein each of said at least two transparent dressings is comprised of an adhesive border portion folded over upon itself to form a polygonal border about a nonadhesive transparent central window, said adhesive border portion and said nonadhesive transparent central window being removably attachable to a common backing by an adhesive surface of said adhesive border portion.

4. The device of claim 1, wherein said dressing folder comprises a sheet of material twice vertically folded along a first vertical fold line 91 and a second vertical fold line so as to define a first vertical portion, a second vertical portion and a third vertical portion of said dressing folder, said vertical portions being vertically hingedly articulated along said first vertical fold line and said second vertical fold line, said sheet of material additionally being once horizontally folded along a horizontal fold line, to define an inferior horizontal portion, having an excisable first segment corresponding to said first vertical portion, a second segment corresponding to said second vertical portion, and an excisable third segment corresponding to said third vertical portion, said second segment being horizontally hingedly articulating along said horizontal fold line when said first segment and said third segment are excised from said sheet of material.

5. The device of claim 1, wherein said inner face of said program comprises a set of inner program panels, each of which contains at least one inner program field of a set of inner program fields.

6. The device of claim 5, wherein said set of inner program panels includes an instructional inner program panel.

7. The device of claim 1, wherein said outer face of said program comprises a set of outer program panels each of which contains at least one outer program field of a set of outer program fields.

8. The device of claim 6, wherein said instructional inner program panel contains at least one inner program field containing instructions and directions for said rotatable selection, preparation and recollection of said actual hemodialysis cannulation sites along said hemodialysis access configuration.

9. The device of claim 5, wherein said set of inner program panels includes an insert inner program panel, said insert inner program panel having said means for said removable receipt of said site preparation card and also having said means for said removable receipt of said at least two transparent dressings.

10. The device of claim 5, wherein said set of inner program panels 15 includes a placeholder inner program panel, said placeholder inner program panel 15c having said means 13 for said removable receipt thereon of said at least one transferably detachable self-adhesive exemplary mnemonic marker 3.

11. The device of claim 10, wherein said placeholder inner program panel contains at least one inner program field containing instructions and directions relevant to using said at least two transferably detachable self-adhesive mnemonic markers.

12. The device of claim 9, wherein said site preparation card comprises a sheet of material having a front face and a back face, said site preparation card being removably receivable by said removable receiving means of said insert inner program panel of said program.

13. The device of claim 12, wherein at least said front face of said site preparation card is laminated with a transparent coating upon which transparent coating said at least two self-adhesive mnemonic markers may be detachably and reversibly transferred from said placeholder inner program panel of said program to said front face of said sheet of card material.

14. The device of claim 12, wherein said site preparation card contains a set of site preparation fields, among which there is included a title site preparation field, a patient data site preparation field, an instruction site preparation field, and an image site preparation field.

15. The device of claim 14, wherein said image site preparation field contains a reproduction of said hemodialysis access configuration.

16. The device of claim 15, wherein said reproduction of said hemodialysis access configuration comprises a reproduction of a surgical arterio-venous fistula.

17. The device of claim 15, wherein said reproduction of said hemodialysis access configuration comprises a reproduction of a straight arterio-venous graft.

18. The device of claim 15, wherein said reproduction of said hemodialysis access configuration comprises a reproduction of a looped arterio-venous graft.

19. The device of claim 16, wherein said reproduction of said arterio-venous fistula appearing in said image site preparation field of said hemodialysis access configuration has superimposed upon it a set of image identification fields, which set of image identification fields includes: an Elbow image identification field; a Wrist image identification field; an Arterial End image identification field proximal to said Wrist image identification field and associated with an Arterial Start image identification field; a Venous End image identification field proximal to said Elbow image identification field and associated with a Venous Finish image identification field; a Midpoint image identification field associated with a slightly distal Arterial Finish image identification field and a slightly proximal Venous Start image identification field; between which Arterial Start image identification field and Arterial Finish image identification field, there is interposed a first approximately linear array of N AVF circles, and between which Venous Start image identification field and Venous Finish image identification field there is interposed a second approximately linear array of N AVF circles.

20. The device of claim 19, wherein N is less than or equal to 6.

21. The device of claim 19, wherein said circles of said first approximately linear array of N AVF circles comprise representative indicators of the actual sites at which an arterial access hemodialysis needle is successively cannulated together with a corresponding venous access hemodialysis needle that is cannulated along a correspondingly numbered circle of said second approximately linear array of N AVF circles, said circles of said first approximately linear array of N AVF circles being numbered in ascending order from 1 to N, the first such circle being about coincident with said Arterial Start image identification field, and the Nth such circle being coincident with said Arterial Finish image identification field, said first approximately linear array of N AVF circles overlying the expanse of said arterio-venous fistula from said Arterial Start image identification field to said Arterial Finish image identification field.

22. The device of claim 19, wherein said circles of said second approximately linear array of N AVF circles comprise representative indicators of the actual sites at which a venous access hemodialysis needle is successively cannulated together with a corresponding arterial access hemodialysis needle that is cannulated along a correspondingly numbered circle of said first approximately linear array of N AVF circles, said circles of said second approximately linear array of N AVF circles being numbered in ascending order from 1 to N, the first such circle being about coincident with said Venous Start image identification field, and the Nth such circle being about coincident with said Venous Finish image identification field, said second approximately linear array of N AVF circles overlying the expanse of said arterio-venous fistula from said Venous Start image identification field to said Venous Finish image identification field.

23. The device of claim 17, wherein said reproduction of said straight arterio-venous graft appearing in said image site preparation field of said hemodialysis access configuration has superimposed upon it a set of superimposed image identification fields, which set of superimposed image identification fields includes: an Elbow image identification field; a Wrist image identification field; an Arterial End image identification field proximal to said Wrist image identification field and associated with an Arterial Start image identification field; a Venous End image identification field proximal to said Elbow image identification field and associated with a Venous Finish image identification field; a Midpoint identification field associated with a slightly distal Arterial Finish image identification field and a slightly proximal Venous Start image identification field; between which Arterial Start image identification field and Arterial Finish image identification field, there is interposed a first approximately linear array of N SAVG circles, and between which Venous Start image identification field and Venous Finish image identification field there is interposed a second approximately linear array of N SAVG circles.

24. The device of claim 23, wherein N is less than or equal to 6.

25. The device of claim 23, wherein said circles of said first approximately linear array of N SAVG circles comprise indicators of the actual sites at which an arterial access hemodialysis needle is successively cannulated together with a paired venous access hemodialysis needle that is cannulated along a correspondingly numbered circle of said second approximately linear array of N SAVG circles, said circles of said first approximately linear array of N SAVG circles being numbered in ascending order from 1 to N, the first such circle being about coincident with said Arterial Start image identification field, and the Nth such circle being coincident with said Arterial Finish image identification field, said first approximately linear array of N SAVG circles overlying the expanse of said arterio-venous fistula from said Arterial Start image identification field to said Arterial Finish image identification field.

26. The device of claim 23, wherein said circles of said second approximately linear array of N SAVG circles comprise indicators of the actual sites at which a venous access hemodialysis needle is successively cannulated together with a paired arterial access hemodialysis needle that is cannulated along a correspondingly numbered circle of said first approximately linear array of N SAVG circles, said circles of said second approximately linear array of N SAVG circles being numbered in ascending order from 1 to N, the first such circle being about coincident with said Venous Start image identification field, and the Nth such circle being coincident with said Venous Finish image identification field, said first approximately linear array of N SAVG circles overlying the expanse of said aterio-venous fistula from said-Venous Start image identification field to said Venous Finish image identification field.

27. The device of claim 18, wherein said reproduction of said looped arterio-venous graft appearing in said image site preparation field of said hemodialysis access configuration has superimposed upon it a set of superimposed image identification fields, which set of superimposed image identification fields includes: an Arterial Side image identification field; a Venous Side image identification field; an Elbow image identification field; a Wrist image identification field; an Arterial Start image identification field proximal to said Elbow image identification field; an Arterial Finish image identification field proximal to said Wrist image identification field between which Arterial Start image identification field and Arterial Finish image identification field, there is interposed a first approximately linear array of N LAVG circles; a Venous Start image identification field proximal to said Wrist image identification field; and a Venous Finish image identification field proximal to said Elbow image identification field, between which Venous Start image identification field and Venous Finish image identification field there is interposed a second approximately linear array of N LAVG circles.

28. The device of claim 27, wherein N is less than or equal to 6.

29. The device of claim 27, wherein each of said numbered circles of said first approximately linear array of N LAVG circles comprises an indicator of an actual site at which an arterial access hemodialysis needle is cannulated together with a paired venous access hemodialysis needle that is simultaneously cannulated at an actual site corresponding to a correspondingly numbered circle of said second approximately linear array of N LAVG circles, said circles of said first approximately linear array of N LAVG circles being numbered, from elbow to wrist, in ascending order from 1 to N, the first such circle being about coincident with said Arterial Start image identification field, and the Nth such circle being about coincident with said Arterial Finish image identification field, said first approximately linear array of N LAVG circles overlying the expanse of said looped arterio-venous graft from Arterial Start image identification field to said Arterial Finish image identification field.

30. The device of claim 27, wherein each of said numbered circles of said second approximately linear array of N circles comprises an indicator of an actual site at which a venous access hemodialysis needle is cannulated together with a paired arterial access hemodialysis needle that is simultaneously cannulated at an actual site corresponding to a correspondingly numbered circle of said first approximately linear array of N circles, said circles of said second approximately linear array of N circles being numbered, from wrist to elbow, in ascending order from 1 to N, the first such circle being about coincident with said Venous Start image identification field and the Nth such circle being coincident with said Venous Finish image identification field, said first approximately linear array of N circles array overlying the expanse of said looped arterio-venous graft from Venous Start image identification field to said Venous Finish image identification field.

31. A method of using a device for the rotatable selection, preparation and recollection of N paired hemodialysis cannulation sites along a hemodialysis access configuration of an ESRD patient, which N paired hemodialysis cannulation sites are sequentially used in the course of N sequential hemodialysis sessions of a hemodialysis cycle of said ESRD patient, comprising the steps of:
a. creating a reproduction of an ESRD patient's hemodialysis access configuration before the commencement of said ESRD patient's hemodialysis cycle;
b. transferring said reproduction of said ESRD patient's hemodialysis access configuration onto an image site preparation field of a site preparation card of said device before the commencement of said ESRD patient's hemodialysis cycle;
c. superimposing a set of image identification fields upon said transferred reproduction of said ESRD patient's hemodialysis access before the commencement of said ESRD patient's hemodialysis cycle;
d. superimposing, before the commencement of said ESRD patient's hemodialysis cycle, a first approximately linear array of circles numbered 1 through N, said first approximately linear array representing a sequence of first hemodialysis access sites on said transferred reproduction of said ESRD patient's hemodialysis access configuration;
e. superimposing, before the commencement of said ESRD patient's hemodialysis cycle, a second approximately linear array of circles of circles numbered 1 through N, said second approximately linear array representing a corresponding sequence of second hemodialysis access sites on said transferred reproduction of said ESRD patient's hemodialysis access configuration;
f. identifying said ESRD patient's hemodialysis configuration in a title site preparation field of said site preparation card of said device, before the commencement of said ESRD patient's hemodialysis cycle;
g. identifying said ESRD patient's patient data in a patient data site preparation field of said site preparation card of said device, before the commencement of said ESRD patient's hemodialysis cycle;
h. providing instruction on the use of said device in an instruction site preparation field of said site preparation card of said device, before the commencement of said ESRD patient's hemodialysis cycle;
i. preplacing upon each circle of said first approximately linear array of circles numbered 1 through N, a first set mnemonic markers, correspondingly numbered 1 through N, before the commencement of said ESRD patient's hemodialysis cycle;
j. preplacing upon each circle of said second approximately linear array of circles numbered 1 through N, a second set mnemonic markers correspondingly numbered 1 through N, before the commencement of said ESRD patient's hemodialysis cycle;
h. defining a hemodialysis session index i={N−(N−x)} for identifying an ith hemodialysis session in a sequence of N hemodialyis sessions, where x=1, 2, . . . N, at the commencement of said ESRD patent's hemodialysis cycle;
i. letting x=1 in i={N−(N−x)} at the commencement of said ESRD patent's hemodialysis cycle.

32. The method of claim 31 comprising the further steps of:
a. removing, prior to an ith hemodialysis session of said N sequential hemodialysis sessions of said ESRD patient's hemodialysis cycle, a first mnemonic marker numbered i from said first approximately linear array of mnemonic markers and a second mnemonic marker also numbered i from said second approximately linear array of mnemonic markers, said first and second mnemonic markers comprising an ith pair of mnemonic markers, said removal of said ith pair of mnemonic markers exposing on said transferred reproduction an ith representative pair of hemodialysis sites selected for cannulation by said ESRD patient or an aide to said ESRD patient;
b. placing said ith pair of mnemonic markers on a pair of placeholders on a placeholder panel of said device prior to said ith hemodialysis session of said N sequential hemodialysis sessions of said ESRD patient's hemodialysis cycle;
c. using a marking device of said device, prior to said ith hemodialysis session of said N sequential hemodialysis sessions of said ESRD patient's hemodialysis cycle, to mark for identification on said ESRD patient's actual hemodialysis access configuraton, an ith marked pair of actual hemodialysis cannulation sites, corresponding to said ith representative pair of hemodialysis sites selected for cannulation by said ESRD patient or an aide to said ESRD patient from said image;
d. coating said ith marked pair of actual hemodialysis cannulation sites with a dollop of anesthetic ointment prior to said ith hemodialysis session of said N sequential hemodialysis sessions of said ESRD patient's hemodialysis cycle;
e. removing a pair of transparent dressings of said device from a dressing folder of said device prior to said ith hemodialysis session of said N sequential hemodialysis sessions of said ESRD patient's hemodialysis cycle;
f. dressing said ith marked pair of actual hemodialysis cannulation sites with said pair of transparent dressings prior to said ith hemodialysis session of said N sequential hemodialysis sessions of said ESRD patient's hemodialysis cycle;
g. submitting said ith marked pair of actual hemodialysis cannulation sites for arterio-venous cannulation during said ith hemodialysis session;
h. recalling said ith marked pair of actual hemodialysis cannulation sites that has been cannulated in said ith hemodialysis session by leaving exposed said ith representative pair of hemodialysis sites selected for cannulation on said transferred reproduction until said ESRD patient or an aide to said ESRD patient selects a succeeding representative pair of hemodialysis sites for cannulation;
i removing said ith pair of mnemonic markers from said pair of placeholders on said placeholder panel of said device when a succeeding representative pair of hemodialysis sites is selected for cannulation by said ESRD patient or an aide to said ESRD patient;
j. restoring said ith pair of mnemonic markers to said ith representative pair of hemodialysis sites previously selected for cannulation;
k. selecting said succeeding representative pair of hemodialysis sites by letting the value of x in i={N−(N−x)} increase by 1 and repeating steps a. through j. until x=N;
l. repeating steps a. through k. for a succeeding hemodialysis cycle of said ESRD patient;
m. teaching said ESRD patient or an aide to said ESRD patient steps a. through m.

33. The method of claim 31 wherein said instruction site preparation field contains instructions for said ESRD patient on using said device to rotatably select, prepare and recall N paired hemodialysis cannulation sites along an AVF hemodialysis access configuration of said ESRD patient.

34. The method of claim 33, wherein N=5.

35. The method of claim 31 wherein said instruction site preparation field contains instructions for said ESRD patient on using said device to rotatably select, prepare and recall N paired hemodialysis cannulation sites along a SAVG hemodialysis access configuration of said ESRD patient

36. The method of claim 34, wherein N=5.

37. The method of claim 31 wherein said instruction site preparation field contains instructions for said ESRD patient on using said device to rotatably select, prepare and recall N paired hemodialysis cannulation sites along a LAVG hemodialysis access configuration of said ESRD patient.

38. The method of claim 37, wherein N=6.
